(19) United States
(12) Patent Application Publication
(10) Pub. No.: US 2018/0256686 A1
(43) Pub. Date: Sep. 13, 2018

(54) Compositions and Methods for Treating Ischemia

(71) Applicant: University of Kentucky Research Foundation, Lexington, KY (US)

(72) Inventors: Gregory J. Bix, Lexington, KY (US); Kathleen Salmeron, Lexington, KY (US)

(21) Appl. No.: 15/849,460

(22) Filed: Dec. 20, 2017

Related U.S. Application Data
(60) Provisional application No. 62/437,205, filed on Dec. 21, 2016.

(51) Int. Cl. A61K 38/20; A61K 9/00; A61P 25/00

(52) U.S. Cl. CPC A61K 38/2006; A61P 25/00; A61K 9/0019

(57) ABSTRACT

Methods of treating ischemia, reducing infarcts and enhancing neuroprotection are provided, and include administration of IL-1α.

### RELATED APPLICATIONS

This application claims priority from U.S. Provisional Patent Application No. 62/437,205, filed Dec. 19, 2016, the entire disclosure of which is incorporated herein by this reference.

### GOVERNMENT INTEREST

This invention was made with government support under Grant No. NIH R21NS085660. The government has certain rights in the invention.

### TECHNICAL FIELD

The presently-disclosed subject matter relates to the treatment of ischemia, including cerebral ischemia and stroke. In particular, embodiments of the presently-disclosed subject matter relate to methods for preventing and/or treating ischemia and potential infarction that utilize IL-1α.

### INTRODUCTION

Tissues deprived of blood and oxygen undergo ischemic necrosis or infarction with possible irreversible organ damage. Stroke is a leading cause of death and disability, with ˜15 million new cases worldwide annually. Ischemic stroke, defined as the blockade of a blood vessel supplying the brain (by a blood clot), comprises ˜80% of all strokes, while the remaining 20% are the result of blood vessel rupture and bleeding (hemorrhagic stroke).

While the initial ischemic event, or primary injury, causes brain tissue damage and cell death, the largest extent of the damage occurs after recanalization of the occluded vessel, also known as reperfusion injury, triggered by excitotoxicity, reactive oxygen species, and subsequently inflammation. The role of inflammation in post-stroke injury has been extensively investigated. The pro-inflammatory cytokine interleukin-1 (IL-1), in particular, has been shown to have a deleterious role after stroke, while blocking its actions is beneficial in pre-clinical and clinical settings (1, 2, 3). IL-1 is known as a “master cytokine” implicated in many neurological conditions, and is the primary mediator of inflammation after stroke. Importantly, although acute inflammation driven by IL-1 is detrimental and contributes to poor outcome after stroke, delayed “low-grade” inflammation is increasingly recognized to be important for brain tissue repair and functional recovery (11). Current novel strategies aiming at reducing IL-1-induced inflammation (using the IL-1 receptor antagonist, IL-1RA, (International Standard Randomized Controlled Trial Number 74236229) may prove beneficial to reduce acute inflammation, but may also have long term reduced efficacy. Therefore, alternative refined and/or complementary strategies to modulate IL-1 for neuroprotection and neurorepair are needed. IL-1 exists as two ligands, Interleukin-1 alpha (IL-1α) and Interleukin-1 beta (IL-1β) that exert their actions by binding to the type-1 IL-1 receptor (IL-1R1) and IL-1R3, a truncated isoform of IL-1R1 that is primarily expressed in neurons (12), while IL-1R2 is a non-signaling decoy receptor.

Interestingly, very little has been done to examine the selective role of each IL-1 isoforms (IL-1α and IL-1β) in ischemic stroke. This assumption presumably stems from the fact that both isoforms bind to the same receptor and have similar biological activity. For a long time, it has been assumed that IL-1α and IL-1β have similar biological activity and mechanism(s) of action (13); however, marked differences between mechanisms of expression and action of these two cytokines exist. Although both isoforms require enzymatic cleavage for generation of the mature proteins, IL-1β is the main secreted isoform, whereas IL-1α remains cytoplasmic driving gene transcription, and several previous studies have demonstrated differential actions of both cytokines in various paradigms of inflammation (14).

Substantial differences in the spatiotemporal expression profile of IL-1α and IL-1β occurs post-stroke; IL-1α is expressed 4 h after stroke (versus 24 h for IL-1β) in and around areas of brain injury and is selectively expressed in microglia (4). In stroke, IL-1α expression precedes that of IL-1β and is significantly less transient than IL-1β (15 h vs 2 h half-life, respectively) (4), suggesting delayed IL-1α expression and action during the chronic phase of inflammation post-stroke. IL-1α expression is predominantly localized in penumbral microglia from 4 to 24 h after stroke. Furthermore, polymorphisms in the human illα abut not illβ gene results in higher instances of vascular malformation and/or higher risk of ischemic stroke (5, 6) suggesting that the two isoforms could play different roles after stroke.

The role of IL-1β in stroke has been fairly well investigated, while the role of IL-1α has remained largely unexplored. Recently, we have demonstrated that IL-1α selectively triggers angiogenesis (an important component of post-stroke neurorepair) (9) in vitro, and increases the production of neuroprotective perlecan LG3 (a 25-kDa protein fragment of the vascular basement membrane component heparan sulfate proteoglycan perlecan) in cells of the neurovascular unit (10). In both studies, IL-1β shares little to none of this activity. IL-1α is also expressed in the periphery after stroke, particularly in platelets, and is released in response to platelet activation (15, 16). Platelets are of particular importance in stroke pathophysiology since they are the key cellular component of thrombi and atherosclerotic plaques, and are involved in the no-reflow mechanism induced by micro-emboli due to their higher circulating number seen in conditions of co-morbidities such as atherosclerosis and infections (known risk factors for stroke) (17). Furthermore, platelets may play a significant role in promoting post-stroke neuroreparative angiogenesis (18, 19). Therefore, IL-1α derives from both central and peripheral sources, although the temporal functional significance of the different sources of IL-1α after stroke is completely unknown.

Apart from the limited data on IL-1α expression mentioned above, very little is known about the precise spatiotemporal expression of IL-1α after stroke, and how this might be influenced by age or gender. This is of importance since studies found that raised inflammatory status associated with aging contributes to poor stroke outcome, whereas raised estrogen levels in cycling female is associated with lower systemic inflammation and contributes to better stroke outcome (20, 21).

Relatedly, healthy aged mice express significantly more IL-1α in several organs (lungs, liver and spleen) compared to their younger counterparts (22), and neurologically normal aged adults (≥60 yrs.) express significantly more activated IL-1α positive brain microglia and tissue IL-1α than younger individuals (23). Furthermore, the sex hormones 17β-estradiol has been shown to increase IL-1α expression in various cell types (24). These observations collectively suggest a potentially significant age and gender effect on IL-1α expression; the later may be relevant to the lower stroke risk and severity seen in young females as compared to young males that exists prior to menopause. The cellular and temporal source of IL-1α in young/aged and male/female could inform whether IL-1α exerts multiple actions, which could be age- or gender-dependent during post-stroke inflammation.

Cerebral ischemia results from decreased blood and oxygen flow implicating one or more of the blood vessels of the brain. In cerebral ischemia, the individual suffers a stroke with sudden development of a focal neurologic deficit and, in most cases, some degree of brain damage. The decreased blood flow may be due to, for example, an occlusion such as a thrombus or embolus, vessel rupture, sudden fall in blood pressure, change in the vessel lumen diameter due to atherosclerosis, trauma, aneurysm, developmental malformation, altered permeability of the vessel wall or increased viscosity or other quality of the blood. Decreased blood flow may also be due to failure of the systemic circulation and severe prolonged hypotension. Ischemic necrosis of the spinal cord may result in sensory or motor symptoms or both that can be referred to cervical, thoracic or lumbar levels of the spine.

Current treatments for ischemia encompass behavioral changes, drug therapy, and/or surgical intervention. Drugs are frequently preferred before resorting to invasive procedures and to provide more immediate relief than long-term behavioral changes. However, current drugs are limited in their effectiveness in preventing infarction.

Similarly, stroke is the fourth leading cause of death in the U.S., and approximately 87% of stroke patients are ischemic, resulting from a lack of blood flow to a part of the brain. Current standard of care for ischemic stroke is rapid reopening of the occluded brain blood vessel with tissue plasminogen activator (t-PA). Unfortunately, t-PA is limited to a brief window of 4.5 hours within symptom onset contributing, along with other factors, to the exclusion of many patients. Furthermore, results from large t-PA trials have been mixed, showing improving recanalization rates, but no overwhelming improvements in outcome. While the speed and efficacy of recanalization (by the use of tissue plasminogen activator or mechanical clot removal) has improved, patients continue to experience poor outcome, and functional recovery is often limited. Unfortunately, despite the promise of many potential neuroprotective therapies, none have thus far succeeded in clinical trials. Thus, new protective and reparative stroke therapies are urgently needed.

Thus, there is a need for a therapeutic agent which can be useful in treating ischemia as well as associated infarction and cell death.

### SUMMARY

Provided herein are methods for preventing and/or treating ischemia in a cell. In some embodiments, a method is provided for preventing ischemia in a cell, wherein the ischemia may be caused by one or more ischemic events. In some embodiment the method includes contacting the cell with IL-1α. In some embodiments, a cell is coated with an IL-1α or is cultured in a solution that includes IL-1α. In other embodiments the cell is within a subject, and contacting includes administering IL-1α to the subject such that a cell within the subject is capable of receiving IL-1α. In some embodiments, such methods can include further administration of IL-1RA at the same time as the administration of IL-1α, or prior to or subsequent to the administration of IL-1α.

Methods for treating ischemia in a subject are disclosed herein, and include administering to a subject in need thereof an effective amount of IL-1α thereby treating the ischemia. In some embodiments the ischemia is caused by an ischemic event such as cerebral ischemia and/or stroke.

In some embodiments, the IL-1α is administered during or after the onset of the ischemia. In some embodiments, the IL-1α is administered about 0.5 to about 4 hours after the onset of the ischemia. In other embodiments, the administration can occur up to several weeks after the onset of ischemia. Alternatively, or in addition to other times of administration, the IL-1α is administered prior to the onset of ischemia, particularly in subjects determined at risk for an ischemic event.

In some instances, the treatment prevents the occurrence of an infarction and/or restores perfusion to organs and tissues. In some instances, the administration reduces the number of apoptopic cells, inflammation and/or newly divided cells is reduced. In some instances, the administration of IL-1α reduces infarct volume and/or peri-infarct expansion. In some instances, administering IL-1α increases perlecan, Cathepsin B mRNA, PTX3, or combinations thereof.

In some instances, IL-1α is administered at a dose of about 0.05 μg/kg to about 5 mg/kg, which can depend on the mode of administration, timing of administration, subject, and other factors. In some embodiments, the administering is performed intravenously or intraarterially.

Methods of reducing infarcts in a subject after ischemic stroke are also provided herein and include administering IL-1α to the subject. In some embodiments, the reduction of infarcts can include the administration of IL-1RA.

In some embodiments, the administration is acute. In one particular method, a method of treating ischemia is provided including the step of administering intravenous IL-1α during the acute phase of injury. In some embodiments, the administering sustains a low grade chronic inflammation. In some instances, the dose of IL-1α is subpathological.

### DESCRIPTION OF THE DRAWINGS

Illustrative aspects of embodiments of the present invention will be described in detail with reference to the following figures wherein:

FIG. 1 includes (A) images of mice contralateral and ipsilateral stratum in vehicle treated and IL-1α treated, scale bar=50 μm; and (B) a plot showing performance of mice treated with vehicle or with IL-1α on a 28 point neurological score. Data are the mean +/−SEM, *p<0.05****p<0.0001.

FIG. 2 includes (A) images of TUNEL stains and DAPI nuclear counterstains of young male mice treated with vehicle or 0.1 ng IL-1α intraarterially (IA) or 1 ng intravenously (IV), after experimental stroke; (B) a chart comparing infarct volume in young male mice after experiment stroke when treated with vehicle or 0.1 ng IL-1α intraarterially (IA) or ing intravenously (IV); and (C) a plot indicating functional recovery after experimental stroke in young male mice control, sham and treated with vehicle or 0.1 ng IL-1α intraarterially (IA) or 1 ng intravenously (IV).

FIG. 3 includes (A) immunoflorescent images of brain parenchyma of mice that underwent experimental ischemic stroke and were treated with vehicle, 0.1 ng or 1 ng IL-1α. Staining was performed with tomato lectin that labels blood vessels and microglial inflammatory cells, which appears green in the figure and with rabbit anti Ki67 that labels all dividing cells, which appears red in the figure. Images are at 10× magnification and scale bars represent 100 p.m; and (B) images of 20 pm brain sections of mice that underwent experimental ischemic stroke. Brain sections were stained using the Millipore ApopTag TUNEL fluorescein (appears green in the figure) kit and mounted using Vector mounting medium with a DAPI cell nuclei counterstain (appears blue in the figure). This is an indicator of cellular death/apoptosis. All pictures were taken at 10× magnification and scale bars represent 100 p.m.

FIG. 4 includes charts of neuronal cell death in vehicle treated of 1 ng/mL IL-1α, 10 ng/mL IL-1α in the context of (A) OGD; and (B) NMDA induced toxicity; (C) includes a chart of neuronal cell death showing PTX3 prevents neuronal cell death in the context of NMDA induced toxicity. Data are the mean +/−SEM (n=9 per group).

FIG. 5 includes charts of fold changes in endothelial cells treated with IL-1α of (A) perlecan and (B) cathepsin mRNA; and (C) a chart showing overexpression of VEGF protein in endothelial cells treated with 0, 1, 10, 100 and 1000 ng/mL PTX3. Data are the mean +/−SEM (n=3 for A and B, n=1 for C).

FIG. 6 includes three potential mechanisms of action of IL-1α. (A) IL-1α crosses the leaky blood-brain barrier and binds to IL-1R3 on neurons. This binding activates AKT kinase and increases potassium channel driven pro-survival pathways leading to increased neuronal survival. (B) IL-1α binds to IL-1R1 and is transported to the nucleus where it drives the expression of Cathepsin B and Perlecan, which are exported to the extracellular matrix and proteolyzed releasing neuroprotective LG3. (C) IL-1α is taken up by astrocytes and neurons and drives the expression of PTX3 which drives upregulation of neuroprotective VEGF in neurons.

FIG. 7 includes a chart of measured infarct volume in wild-type and perlecan knockout mice treated with PBS or IL-1α. Data are the mean +/−SEM (n=5 per group), #### p<0.0001 compared to Pln KO treated animals, ****p<0.0001 compared to WT controls.

FIG. 8 includes differential expression of IL-1α at 1 day and 14 days following stroke. Images in column A show colocalization of IL-1α with microglia (Iba-1) at 1 day after stroke. Images in column B show colocalization of IL-1α with platelets (CD41) at 14 days after stroke. Images in column C show the lack of colocalization of IL-1α with astrocytes (GFAP) after stroke. Images A-C are quantified in chart (D) showing how IL-1α is expressed at different times in microglia and platelets after stroke.

FIG. 9 includes comprehensive charts showing IL-1α imparts direct neuroprotection in vitro. Both charts show the dose response of primary cortical neurons to IL-1α in the context of normoxia and of oxygen-glucose deprivation. IL-1α preserves primary neuron viability when treated with 1 ng/mL and 10 ng/mL.

FIG. 10 charts how IL-1α is hemodynamically safe up to 1×10⁵ times the effective dose investigated, as measured by core temperature, pulse distension, heart rate and infarct volume.

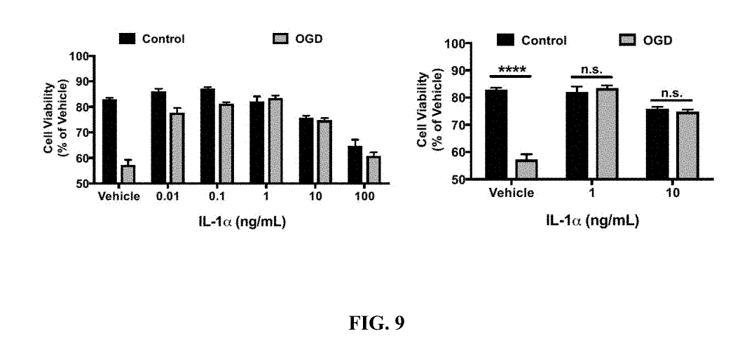

FIG. 11 includes images of intraarterially and intravenousely administered IL-1α and effect on apoptosis, as well as charts of the administration, showing how treatment reduces apoptotic cell death following stroke.

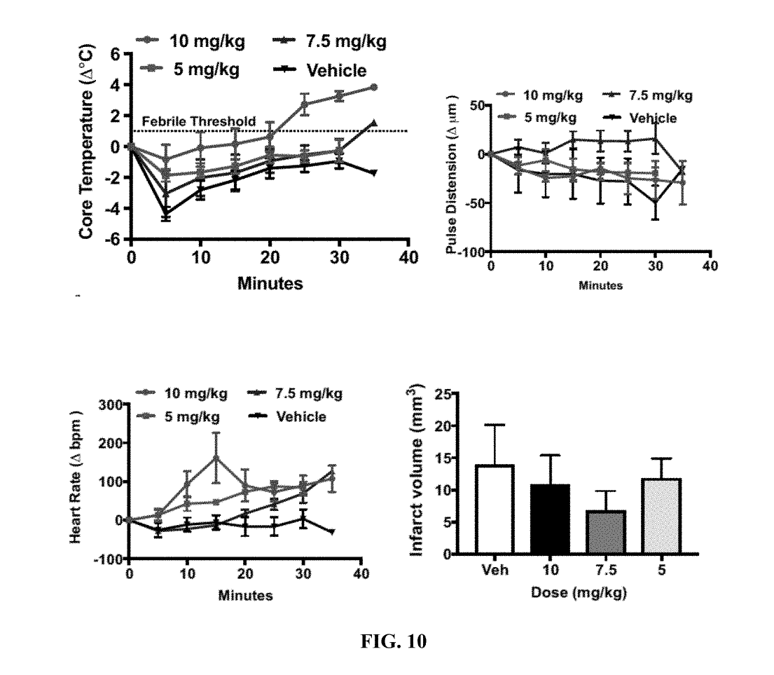

FIG. 12 includes images and chart showing intra-arterial administration of IL-1α reduces intra-parenchymal inflammation. IV and IA IL-1α administration decreases microglial activation (CD11b) within the stroke-injured area.

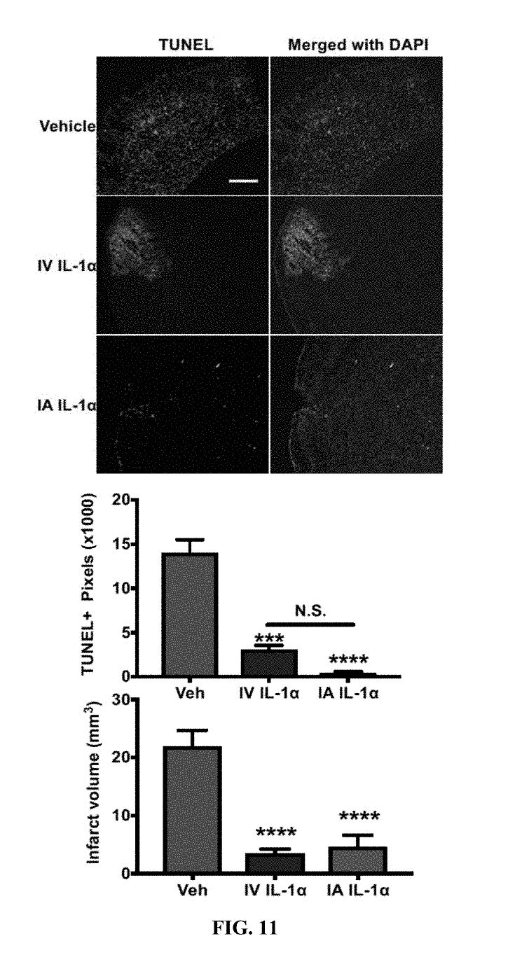

FIG. 13 includes a chart of motor function assessed by open field behavioral testing following stroke for both intravenously and intra-arterially administered IL-1α through post-stroke day 7. IV and IA administration of IL-1α significantly protects against functional loss due to stroke.

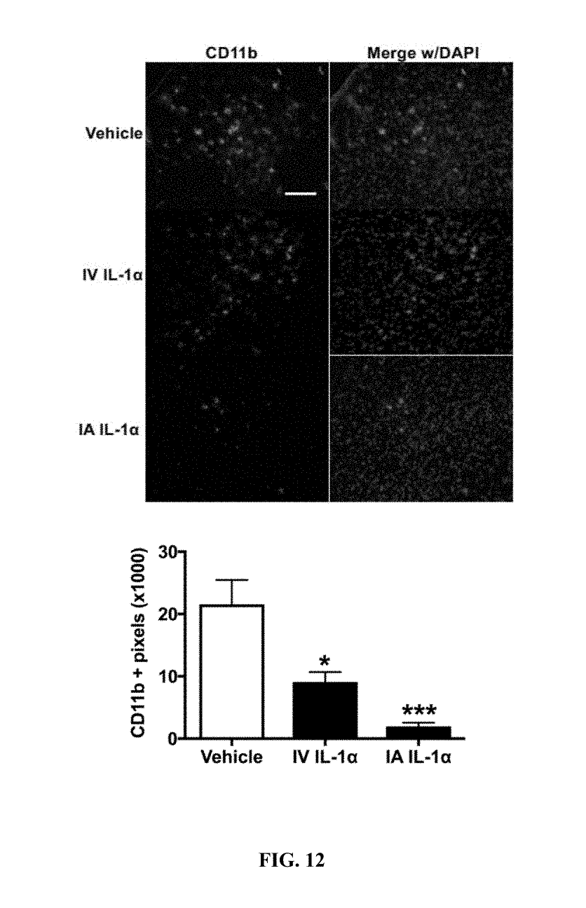

FIG. 14 includes images and a chart of IL-1α imparting neuroprotection through perlecan. Mice lacking intact perlecan do not receive the same degree of neuroprotection as WT mice suggesting that perlecan is necessary for IL-1α's protective action.

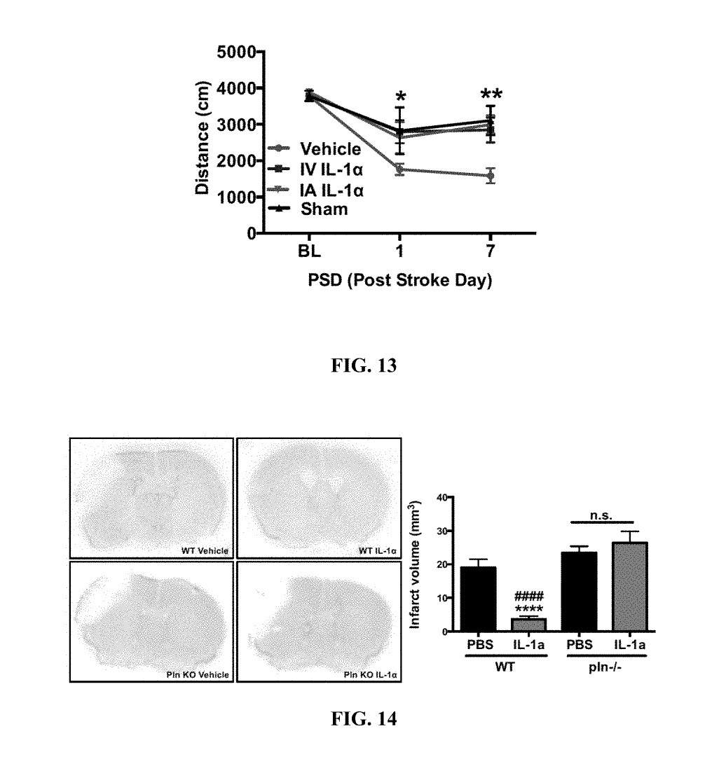

FIG. 15 includes images and chart showing IL-1α treated animals have less overall tissue damage compared to vehicle controls.

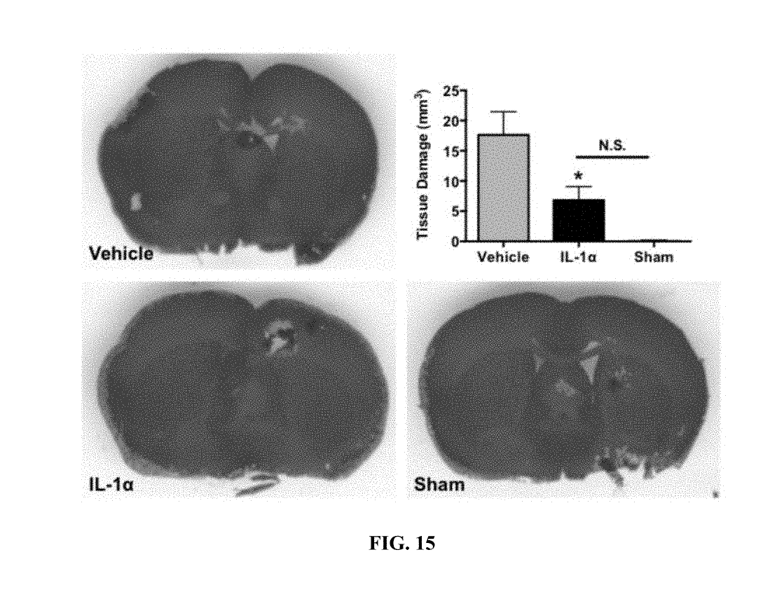

FIG. 16 includes charts showing animals treated with IL-1α perform better on several behavioral tests than vehicle controls.

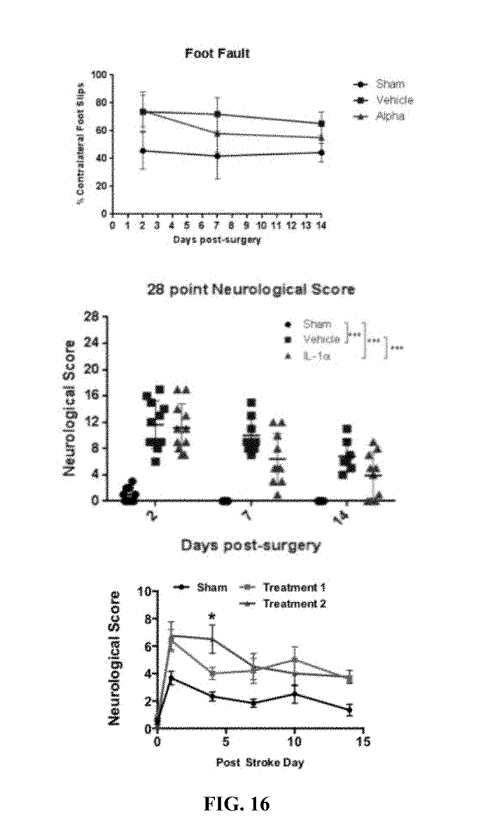

FIG. 17 includes images and charts showing animals have greater vascular density and increased vascular activation after IL-1α administration than vehicle controls.

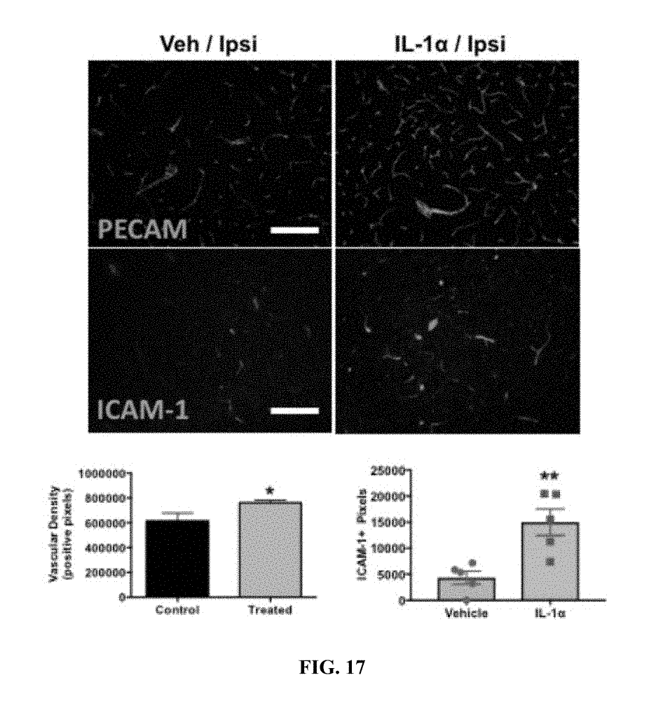

FIG. 18 includes images and a chart showing IL-1α increases endothelial cell division and proliferation in the peri-infarct region compared to controls.

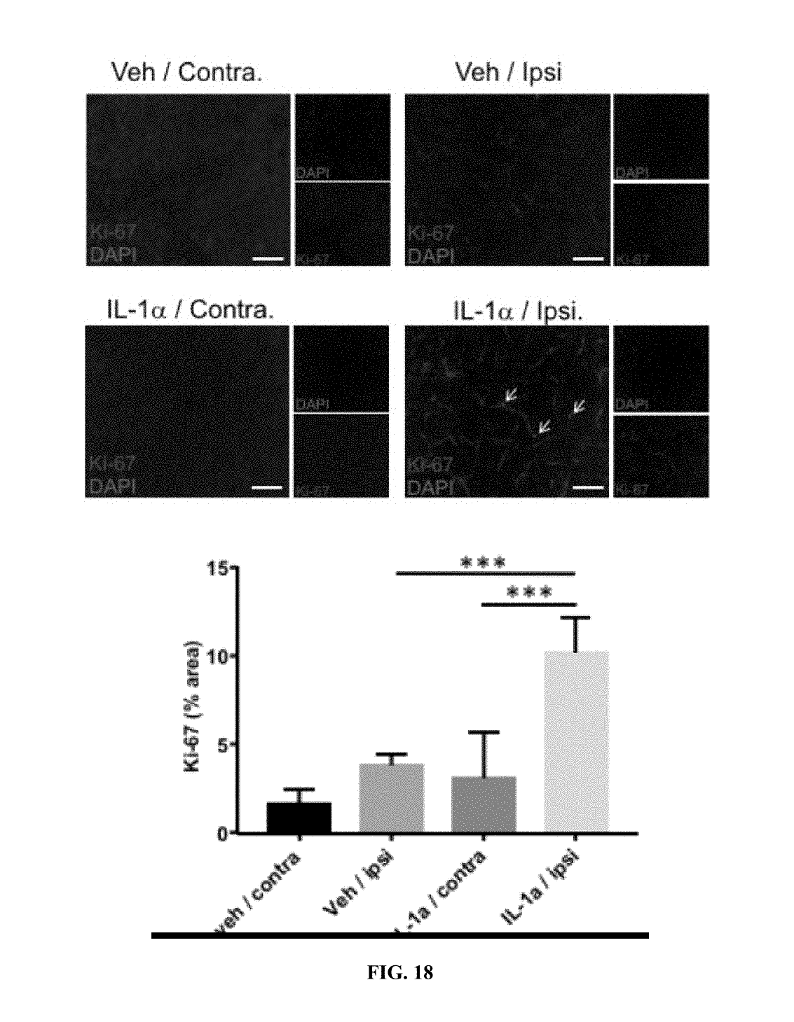

FIG. 19 includes images and charts of IL-1α increasing VEGFR2 expression in the peri-infarct region compared to controls.

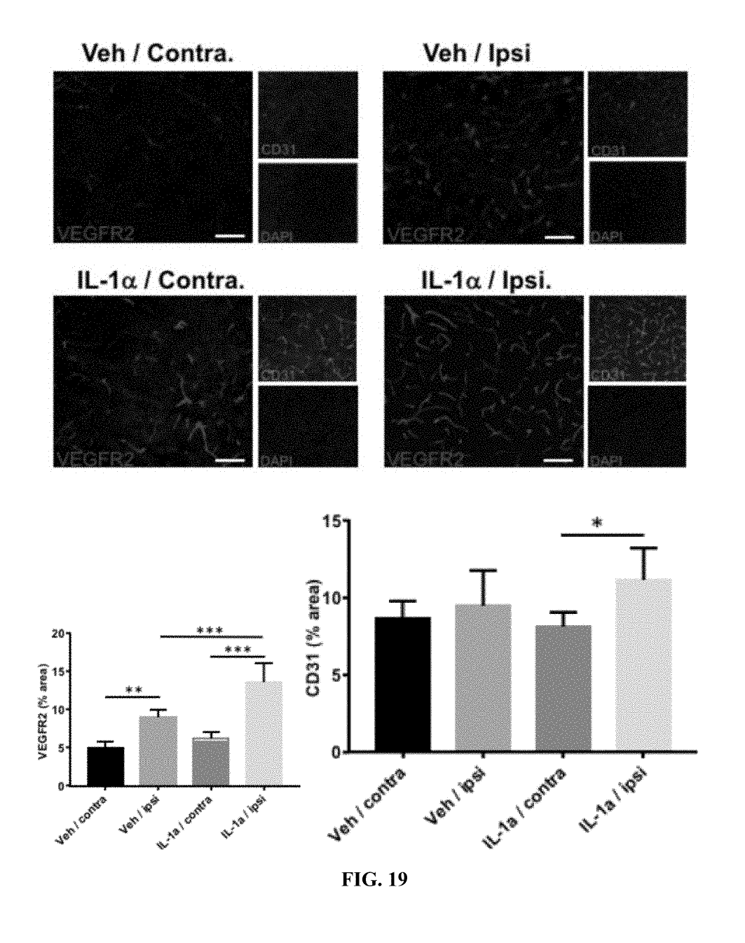

FIG. 20 includes (A) plot of body weight over time after surgery for IL-1α treated mice showing that IL-1α does not cause significant weight loss over time (B) charts measuring lesion volume at 14 days; and (C) chart measuring % increase in IgG staining in IL-1α MCaO mice.

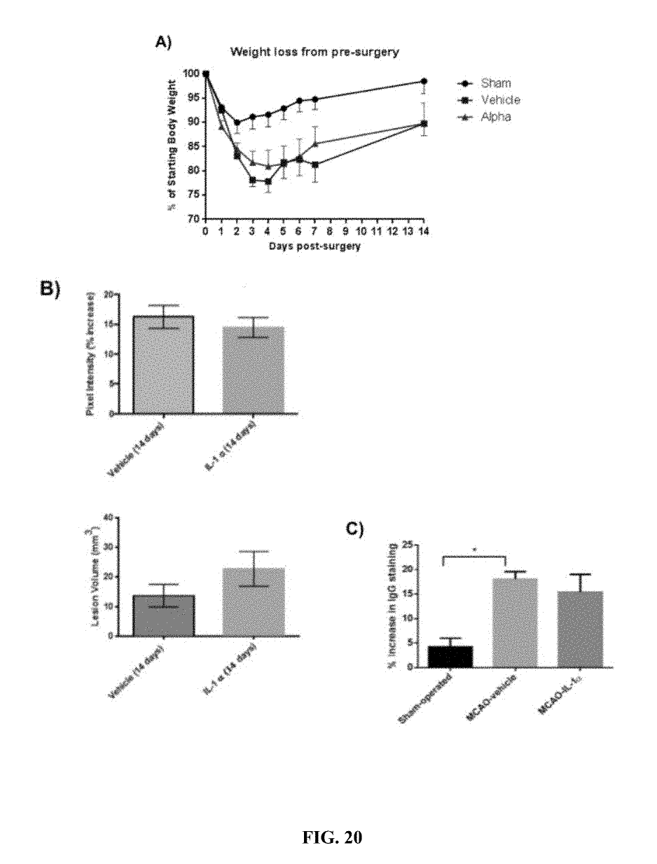

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding, and no unnecessary limitations are to be understood therefrom.

The presently disclosed subject matter is directed to preventing and/or treating ischemia with methods that involve use of IL-1α. As is known in the art, ischemia can lead to infarction in a subject. Therefore, the presently-disclosed subject matter further relates to methods for preventing and/or treating an infarction in a subject, and such methods involve the use of IL-1α.

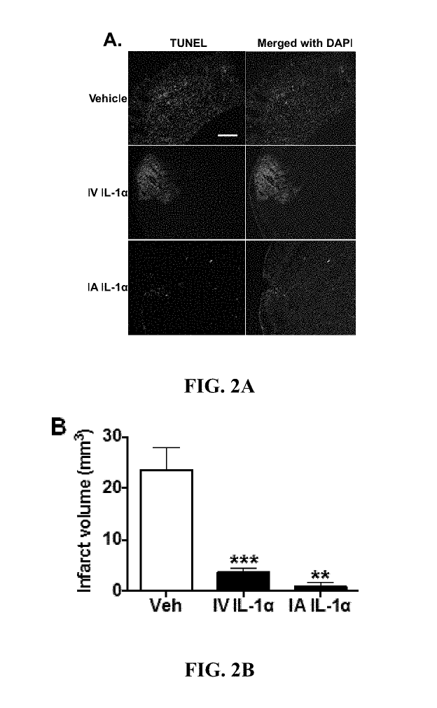

As disclosed herein acutely administered (IV) IL-1α is profoundly neuroprotective after experimental stroke (FIG. 2). Taken together with the chronic actions of IL-1α on angiogenesis, the presently disclosed subject matter suggests that IL-1α has a sustained role during both the acute and chronic phases of stroke. IL-1α is rapidly and persistently elevated in the brain after stroke and has important, potentially beneficial roles in neuroprotection and repair. Based on this discovery, further investigations into intra-arterial (IA) administration suggest that it is an even more effective route for IL-1α administration. Additionally, because of the neuroprotective effects of IL-1α, the presently disclosed methods could find use in other applications where neuroprotection is needed, such as, for example, traumatic brain injury.

In some embodiments, a method is provided for preventing ischemia in a cell, wherein the ischemia may be caused by one or more ischemic events. In some embodiment the method includes contacting the cell with IL-1α. The term “contacting” as used herein refers to any means by which IL-1α is brought into sufficient proximity and/or in direct contact with a cell such that the cell is capable of receiving the IL-1α. For instance, in some embodiments contact refers to coating a cell with an IL-1α. In other embodiments contact refers to culturing a cell in a solution that includes IL-1α. In other embodiments the cell is within a subject, and contact refers to administering an IL-1α to the subject such that a cell within the subject is capable of receiving IL-1α. In some embodiments, such methods can include further administration of IL-1RA at the same time as the administration of IL-1α, or prior to or subsequent to the administration of IL-1α. In some embodiments, the IL 1RA can be administered immediately before or up to 1, 2, 3, 4, 5, 6, 7, 8, 9, 10, 11, 12, 13, 14, 15, 16, 17, 18, 19, 20, 21, 22, 23, or 24 hours prior to administration of IL-1α. In some embodiments, the IL 1RA can be administered immediately after or up to 1, 2, 3, 4, 5, 6, 7, 8, 9, 10, 11, 12, 13, 14, 15, 16, 17, 18, 19, 20, 21, 22, 23, or 24 hours after administration of IL-1α.

The term “preventing” as used herein refers to the characteristic of reducing or eliminating ischemia as well as the side effects associated with ischemia, which can include infarction. The term “preventing” does not imply a particular degree of reduction or elimination of ischemia. Likewise, the term “preventing” does not imply that infarction due to ischemia is eliminated. Instead, the term “preventing” refers to reducing ischemia as well as side effects thereof, including potentially infarction, by about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% relative to a control that has not been contacted or treated with an IL-1α.

With respect to methods for preventing ischemia in a cell, in some embodiments the cell is a brain cell. In other embodiments the cell is part of a particular tissue, and the method includes preventing ischemia in the cell(s) of the tissue. In this respect, the term “tissue” is used herein to refer to a population of cells, generally consisting of cells of the same kind that perform the same or similar functions. The types of cells that make the tissue are not limited. In some embodiments tissue is part of a living organism, and in some embodiments tissue is tissue excised from a living organism or artificial tissue. In some embodiments tissue can be part of an organ, wherein the term “organ” refers to a part of a subject which is composed of several tissues and adapted to perform a specific function or functions, such as the brain.

The presently-disclosed subject matter also relates to methods for treating ischemia in a subject. In some embodiments the method comprises administering to the subject an effective amount of IL-1α. In some embodiments the method further comprises administering to the subject an effective amount IL-1RA.

The term “administering” refers to any method of providing an IL-1α and/or pharmaceutical composition thereof to a subject. Such methods are well known to those skilled in the art and include, but are not limited to, oral administration, transdermal administration, nasal administration, intracerebral administration, and administration by injection, which itself can include intravenous administration, intra-arterial administration, intramuscular administration, subcutaneous administration, intravitreous administration, intracameral (into anterior chamber) administration, and the like. Administration can be continuous or intermittent. In various aspects, a preparation can be administered therapeutically; that is, administered to treat an existing disease or condition (e.g., ischemia, infarction, etc.). In other instances a preparation is administered prophylactically; that is, administered to prevent or treat a disease or condition that may otherwise develop. In some embodiments, the administration is intra-arterially or intravenously.

As used herein, the terms “effective amount” and “therapeutically effective amount” are used interchangeably and mean a dosage sufficient to provide treatment. The exact amount that is required will vary from subject to subject, depending on the species, age, and general condition of the subject, the particular carrier or adjuvant being used, mode of administration, and the like. As such, the effective amount will vary based on the particular circumstances, and an appropriate effective amount can be determined in a particular case by one of ordinary skill in the art using only routine experimentation.

In some instances an effective amount is determined relative to the weight of a subject, and can be selected from dosages of about 0.001 μg/kg to about 5 mg/kg when administered intraarterially and about 0.01 μg/kg to about 5 mg/kg when administered intravenously. In some instances, the dosages can be selected from 0.001 μg/kg, 0.002 μg/kg, 0.003 μg/kg, 0.004 μg/kg, 0.005 μg/kg, 0.006 μg/kg, 0.007 μg/kg, 0.008 μg/kg, 0.009 μg/kg, 0.010 μg/kg, 0.015 μg/kg, 0.02 μg/kg, 0.025 μg/kg, 0.03 μg/kg, 0.035 μg/kg, 0.04 μg/kg, 0.045 μg/kg, 0.05 μg/kg, 0.055 μg/kg, 0.06 μg/kg, 0.065 μg/kg, 0.07 μg/kg, 0.075 μg/kg, 0.08 μg/kg, 0.085 μg/kg, 0.09 μg/kg, 0.095 μg/kg, 0.1 μg/kg, 0.15 μg/kg, 0.2 μg/kg, 0.25 μg/kg, 0.3 μg/kg, 0.35 μg/kg, 0.4 μg/kg, 0.45 μg/kg, 0.5 μg/kg, 0.55 μg/kg, 0.6 μg/kg, 0.65 μg/kg, 0.7 μg/kg, 0.75 μg/kg, 0.8 μg/kg, 0.85 μg/kg, 0.9 μg/kg, 0.95 μg/kg, 1 μg/kg, 2 μg/kg, 3 μg/kg, 4 μg/kg, 5 μg/kg, 6 μg/kg, 7 μg/kg, 8 μg/kg, 9 μg/kg, 10 μg/kg, 11 μg/kg, 12 μg/kg, 13 μg/kg, 14 μg/kg, 15 μg/kg, 16 μg/kg, 17 μg/kg, 18 μg/kg, 19 μg/kg, 20 μg/kg, 21 μg/kg, 22 μg/kg, 23 μg/kg, 24 μg/kg, 25 μg/kg, 26 μg/kg, 27 μg/kg, 28 μg/kg, 29 μg/kg, 30 μg/kg, 31 μg/kg, 32 μg/kg, 33 μg/kg, 34 μg/kg, 35 μg/kg, 36 μg/kg, 37 μg/kg, 38 μg/kg, 39 μg/kg, 40 μg/kg, 41 μg/kg, 42 μg/kg, 43 μg/kg, 44 μg/kg, 45 μg/kg, 46 μg/kg, 47 μg/kg, 48 μg/kg, 49 μg/kg, 50 μg/kg, 0.055 mg/kg, 0.06 mg/kg, 0.065 mg/kg, 0.07 mg/kg, 0.075 mg/kg, 0.08 mg/kg, 0.085 mg/kg, 0.09 mg/kg, 0.095 mg/kg, 0.1 mg/kg, 0.15 mg/kg, 0.2 mg/kg, 0.25 mg/kg, 0.3 mg/kg, 0.35 mg/kg, 0.4 mg/kg, 0.45 mg/kg, 0.5 mg/kg, 0.55 mg/kg, 0.6 mg/kg, 0.65 mg/kg, 0.7 mg/kg, 0.75 mg/kg, 0.8 mg/kg, 0.85 mg/kg, 0.9 mg/kg, 0.95 mg/kg, 1 mg/kg, 1.5 mg/kg, 2 mg/kg, 2.5 mg/kg, 3 mg/kg, 3.5 mg/kg, 4 mg/kg, 4.5 mg/kg and 5 mg/kg.

The term “subject” is used herein to refer to a target of administration, which optionally displays symptoms related to a particular disease, pathological condition, disorder, or the like. Thus, in some embodiments a subject refers to a target that displays symptoms of ischemia and/or infarction. The subject of the herein disclosed methods can include both human and animal subjects. A subject can be, but is not limited to, vertebrates, such as mammals, fish, birds, reptiles, or amphibians. More specifically, the subject of the herein disclosed methods can include, but is not limited to, a human, non-human primate, cat, dog, deer, bison, horse, pig, rabbit, dog, sheep, goat, cow, cat, guinea pig, or rodent. The term does not denote a particular age or sex. Adult and newborn subjects, as well as fetuses, whether male or female, are intended to be covered. The term “subject” includes human and veterinary subjects.

The terms “treat,” “treatment,” and the like refer to the medical management of a subject with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative (prophylatic) treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

In some methods for treating ischemia, the ischemia is caused by a particular ischemic event. In some instances, the ischemia is caused at least in part by an ischemic event selected from cerebral ischemia, stroke, and a combination thereof. In some embodiments the IL-1α is administered one or more times during or after the onset of ischemia and/or during or after an ischemic event. In this respect, in some embodiments IL-1α is administered one or more times during or after the onset of two or more distinct ischemic events, and therefore the present methods are not limited to a single administration of IL-1α. In such embodiments, IL-1α can optionally be administered immediately after or about 0.25, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, or 72 hours after the onset of the ischemic.

As discussed above, treatment can be preventative (prophylactic) in some instances. Accordingly, in some embodiments of the presently-disclosed treatment methods, the IL-1α is administered prior to the onset of ischemia and/or prior to an ischemic event. In some embodiments the IL-1α is administered prior to each of one or more separate onsets of ischemia and/or ischemic events. For example, in some embodiments, the IL-1α is administered prior to an event or situation in which there is a high risk of an ischemic event, e.g., a planned surgical procedure with a high risk of an ischemic event given the nature of the procedure and/or the subject.

In some embodiments administration of IL-1α to a subject, prior to, during, and/or after the onset of ischemia, can prevent (i.e., 1-100% reduction relative to control) the occurrence of infarction in the subject. Alternatively or additionally, in some embodiments administration of IL-1α to a subject, prior to, during, and/or after the onset of ischemia, can restore perfusion to tissues and organs in the subject.

Further still, the presently-disclosed subject matter relates to methods for preventing an infarction in a subject. In some embodiments, a method for preventing an infarction in a subject comprises administering an effective amount of IL-1α. In some embodiments the IL-1α is administered with IL-1RA.

In some embodiments of methods for preventing an infarction in a subject, prior to administering IL-1α, the subject is first diagnosed and/or prognosed as having ischemia. In some instances the subject can be diagnosed as having ischemia that has already led to an infarction in the subject. Administration of IL-1α prior to an ischemic event, whether or not a subject has been prognosed as being at risk for ischemia, could therefore serve as a preventative treatment for ischemia, and potentially infarction.

The terms “diagnose” and the like as used herein refer to methods by which the skilled artisan can estimate and even determine whether or not a subject is suffering from a given disease or condition, such as ischemia. Along with diagnosis, clinical “prognosis” or “prognosticating” is also an area of great concern and interest, and the terms “prognose” and the like refer to act of determining the relative risk associated with particular conditions in order to plan the most effective therapy. If an accurate prognosis can be made, appropriate therapy, and in some instances less severe therapy or more effective therapy, for the subject can be chosen. For instance, in some embodiments of the presently disclosed subject matter, a subject that is prognosed as having ischemia can have IL-1α administered in order to prevent the potential ischemia from developing.

In some embodiments, the administration of IL-1α alone or in combination with another therapy such as IL-1RA reduces the number of apoptopic cells, inflammation and/or newly divided cells. In some embodiments such administration reduces infarct volume and/or peri-infarct expansion. In some embodiments the administration of IL-1α increases perlecan, Cathepsin B mRNA, PTX3, or combinations thereof. In some embodiments, measurements of such indicators can determine effectiveness of treatment, need for additional treatment, and/or diagnosis/prognosis in a subject. In such instances, effectiveness can be determined relative to a control, wherein IL-1α is not administered and/or placed in contact with the target. In some embodiments, a decrease or increase relative to a control can be about a 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% decrease or increase.

Those of ordinary skill in the art will recognize factors and methods for diagnosing and/or prognosing a subject with ischemia. Factors that can contribute to a diagnosis and/or prognosis of ischemia in a subject include, but are not limited to, hypercholesterolemia, electrocardiogram (EKG) changes associated with a risk of or the presence of ischemia (e.g., peaked or inverted T-waves or ST segment elevations or depression in an appropriate clinical context), sedentary lifestyles, angiographic evidence of partial coronary artery obstruction, evidence of a cerebrovascular accident CVA, and other clinical evidence of ischemia.

In some embodiments, the administration of IL-1α is acute. In one particular method, a method of treating ischemia is provided including the step of administering intravenous IL-1α during the acute phase of injury. In some embodiments, the administering sustains a low grade chronic inflammation. In some instances, the dose of IL-1α is subpathological. Sustaining low grade chronic inflammation can be achieved by subpathological dosing and can be monitored, if desired, through various tests for chronic inflammation known in the art such as C Reactive Protein or IL-6 measurements.

### EXAMPLES

The presently-disclosed subject matter is further illustrated by the following specific but non-limiting examples. The examples may include compilations of data that are representative of data gathered at various times during the course of development and experimentation related to the presently-disclosed subject matter. Furthermore, some of the examples described herein may be prophetic examples.

### Example 1

IL-1α is investigated for therapeutic use by intra-atrial (IA) or intravenous (IV) administration for neuroprotection after stroke. In particular, this Example demonstrates IL-1α is neuroprotective when acutely administered at low, subpathological doses IV or IA after stroke, and that IA administration may be as or more beneficial without causing hemodynamic changes. The example also provides prophetic plans to further investigate the neuroprotection studies.

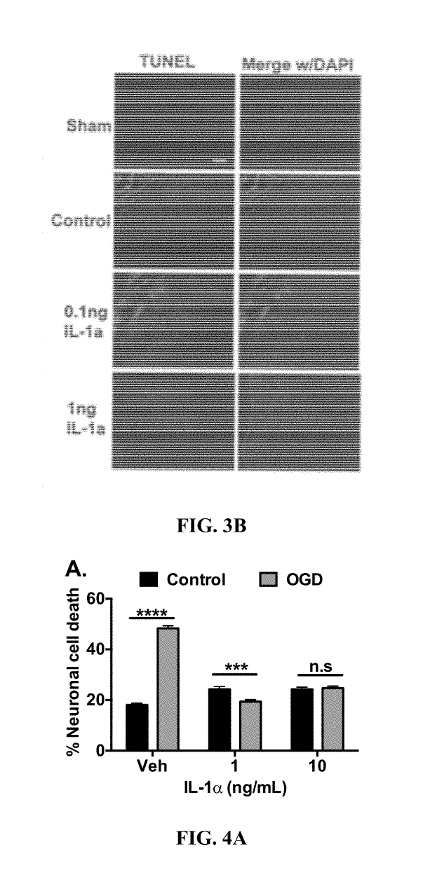

Blocking IL-1 actions using IL-1RA is currently being considered as an anti-inflammatory therapeutic approach targeting the detrimental acute phase of inflammation (1). While blockade of IL-1β is beneficial due to its pro-inflammatory and pro-apoptotic activity, our published (9) and preliminary results highlight the possibility that blocking IL-1α during both the acute and chronic phase of inflammation may be detrimental. To this end, a refined time window of IL-1RA administration or selective inhibition of IL-1β to favor the beneficial effects of IL-1α could be achieved by co-administering IL-1RA followed by chronic low IL-1α regimens. In support of this, in vitro studies described herein and recently published studies found that IL-1α has a beneficial effect on endothelial cells (9) and also results in the release of neuroprotective LG3 from cells of the neurovascular unit (NVU) (10, 30). The current study provides evidence showing that acute administration of IL-1α after stroke results in smaller infarcts, decreased brain tissue damage 7 days after stroke, and better functional recovery compared to vehicle-treated mice (FIG. 2). Although IL-1α is known for its pro-inflammatory effect when expressed at pathological levels, IL-1α has many concentration-specific pleiotropic actions as it also acts as a growth factor, a differentiation factor, and a maturation factor when expressed at low physiological or sub-pathological levels (see (31) for review). Importantly, numerous in vitro and ex vivo studies have reported that IL-1β is neuroprotective against various paradigms of neuronal injury, including excitotoxicity, hypoxia and oxygen-glucose deprivation (OGD) (32,33), and our preliminary data demonstrate that IL-1α is highly neuroprotective against N-methyl-D-aspartate (NMDA)- and OGD-induced neuronal cell death (FIG. 4).

It is believed that administration of low sub-pathological doses of IL-1α is highly neuroprotective after ischemic stroke. Preliminary data suggests that sub-pathological doses of IL-1α, administered IV or IA, results in better functional recovery, smaller infarcts and less peri-infarct expansion all with minimal, non-lethal side effects (FIG. 2). It is proposed that a pro-inflammatory cytokine (i.e. IL-1α) could be used therapeutically (alone or potentially in combination with IL-1RA in future studies) to induce neuroprotection introducing the concept of protective neuroinflammation.

Three month old male C57BL6 mice underwent Middle Cerebral Artery Occlusion (MCAo) for 1 h, and immediately upon reperfusion were treated with IL-1α IV or IA (0.1 ng or 1 ng, respectively) or vehicle (0.9% NaCl+0.1% low endotoxin BSA). Blood pressure and heart rate were monitored with the MouseOx Pulse Oximetry System (Starr Life Sciences).

Results: Mice receiving vehicle treatment experienced a significant increase in blood pressure and a drop in heart rate (data not shown) that is similar to what is typically seen in a clinical presentation of stroke (34). Interestingly, while IV IL-1α-treated mice experienced a similar, somewhat greater, drop in heart rate, they did not experience the same increase in blood pressure (data not shown). This suggests that systemically IV-administered IL-1α could stabilize blood pressure, perhaps by causing vasodilation, which could also be beneficial. Importantly, mice that received IL-1α IA did not experience the transient hemodynamic changes that were seen in the cohort given IV IL-1α (data not shown). Furthermore, mice treated with IL-1α (IV or IA) performed significantly better on tracked, free-movement tests compared to controls that were not significantly different from sham animals (FIG. 2C).

Upon sacrifice, brains were flash frozen and stained for markers of cell health and morphology (H&E and cresyl violet) as well as markers of astrocyte activation (GFAP) and apoptosis (TUNEL). We found that IV and IA IL-1α treated animals showed a significant reduction in cellular dysmorphia, significantly fewer activated astrocytes (data not shown) and apoptotic cells and a significantly reduction in infarct volume compared to vehicle controls (FIG. 2B). Unexpectedly, mice that received IL-1α IA showed an even greater reduction in apoptotic cells and infarct volume than those receiving the IL-1α IV (FIG. 2A, 2B). Taken together, these results suggest that IL-1α is neuroprotective when acutely administered at low, subpathological doses IV or IA after stroke, and that IA administration may be as or more beneficial without causing hemodynamic changes (further suggesting that such hemodynamic changes are not primarily responsible for IL-1α therapeutic benefits).

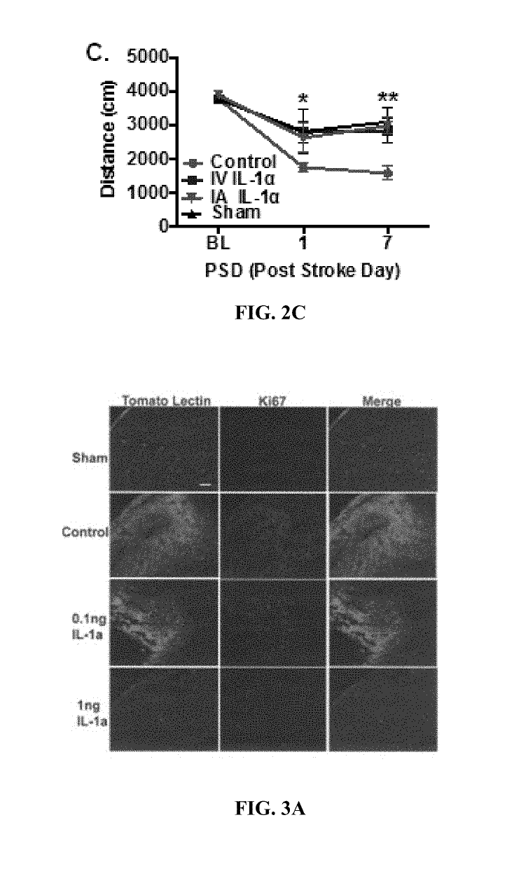

In further investigation, three month old C57BI6 mice underwent transient tandem CCA/MCA occlusion (experimental ischemic stroke) for one hour and treated as labeled above via tail vein injection (n=6 mice per treatment group) immediately after brain reperfusion. After 7 days, the animals were euthanized, their brains removed, frozen and sectioned (20 p.m sections) with a cryostat. As provided in FIG. 3A, results indicate that stroke in control animals caused a significant increase in the number of inflammatory and newly divided cells infiltrating the brain parenchyma that was prevented, in a dose-dependent fashion, by administration of IL-1α. FIG. 3B shows the results of TUNEL fluoresecein and DAPI cell nuclei counterstain that indicates cellular death/apoptosis. FIG. 3B indicates that stroke in control animals caused significant cell death/apoptosis in the brain that was prevented by IL-1α in a dose dependent fashion.

Mechanisms of neuroprotection triggered by IL-1α: IL-1α is directly neuroprotective against NMDA-induced and OGD-induced neuronal cell death in cultured primary neurons. IL-1α stimulates brain angiogenesis in vitro via actions through IL-1R1, but also it has been recently found that IL-1α is profoundly neuroprotective when administered acutely IV and IA at low sub-pathological doses (FIG. 2), as described herein. The mechanisms of action of IL-1α are not well understood and not well studied in stroke. Importantly, IL-1α is known to readily cross the BBB and could therefore have direct and indirect effects on multiple cell types of the neurovascular unit when administered IV or IA (39). Indeed, the previously published findings, combined with this recent data point toward three possible mechanisms of neuroprotection triggered by IL-1α.

The direct neuroprotective effect of IL-1α on neurons after stroke will be investigated. In support of this, the data provided here show IL-1α is directly neuroprotective against NMDA-induced and OGD-induced neuronal cell death in cultured primary neurons (FIG. 4) and, without being bound by theory, believe this supports IL-1α triggers activation of the neuroprotective PI3K/Akt signaling pathway by binding to the neuronal specific IL-1R3 (12) rather than IL-1R1. Indeed, some, if not all, neuroprotective actions of IL-1 are believed to be triggered independently of the classical receptor IL-1R1 (40), and we have reported IL-1R1-independent IL-1 actions in the brain (41). Secondly, the possibility that IL-1α is indirectly neuroprotective via interaction with the endothelium to stimulate the generation of neuroprotective perlecan LG3 will be investigated. This possibility is supported by our previous work demonstrating that IL-1α stimulates cultured primary mouse brain endothelial cells to generate LG3, and that administered LG3 is neuroprotective after OGD in vitro (30).

IL-1α and expression of neuroprotective and angiogenic factors: Procedures were also performed to characterize how IL-1α affects the expression of neuroprotective and angiogenic factors.

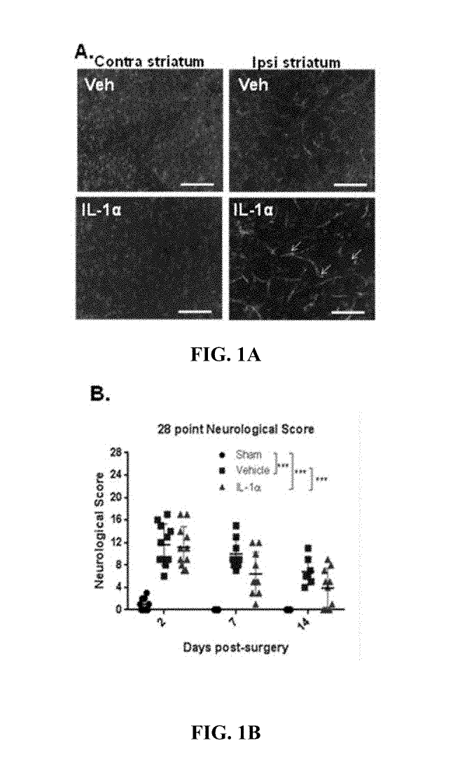

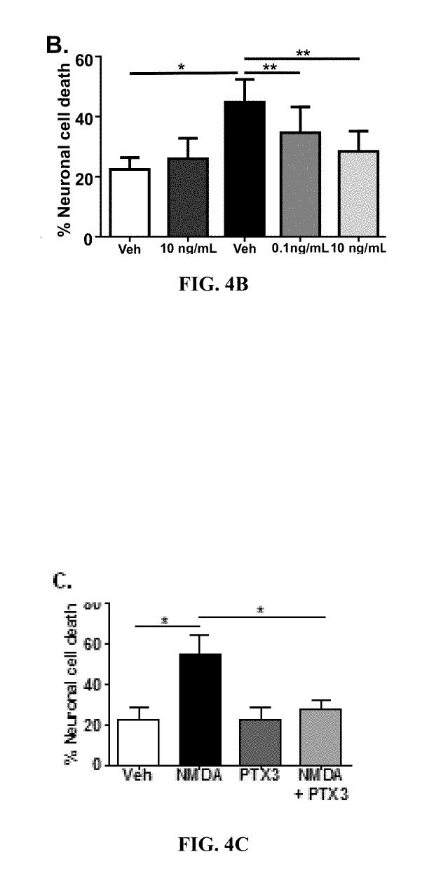

We have demonstrated that IL-1α mRNA levels are significantly elevated 7 days after stroke while protein levels remain significantly elevated for up to 6 weeks (data not shown) (9). This chronic expression of IL-1α appears to induce important delayed repair mechanisms since low dose of IL-1α administered IV at post-stroke day (PSD) 3 induces brain angiogenesis two weeks after the onset of stroke (FIG. 1A). Importantly, IL-1α-induced angiogenesis correlated with significant motor recovery (FIG. 1B) demonstrating the beneficial therapeutic potential of IL-1α for brain repair and functional recovery after stroke. IL-1α increases mRNA levels of perlecan and the LG3-generating (from perlecan) protease cathepsin B in cultured brain endothelial cells (FIGS. 5A and B). These results suggest the possibility of a feedback loop in which IL-1α increases both the production of perlecan and the necessary protease to generate LG3 from perlecan, and that IL-1α increases the expression of PTX3 in neurons and astrocytes that is then in turn neuroprotective by increasing VEGF and LG3 expression in neurons. In support of this, previously published data showed that IL-1α is a key inducer of PTX3 in glial and neuronal cells (7). Moreover, the data disclosed herein now show that PTX3 is highly neuroprotective (FIG. 4C), and demonstrate that PTX3 also induced high expression of neuronal VEGF (FIG. 5C), which can be neuroprotective and pro-angiogenic (8). In further support of a potentially beneficial role of IL-1α after stroke, we have demonstrated that IL-1α induces neurons, brain endothelial cells and astrocytes to generate the LG3 neuroprotective protein fragment of the extracellular matrix component perlecan, a prominent component of the blood-brain barrier (BBB) (10).

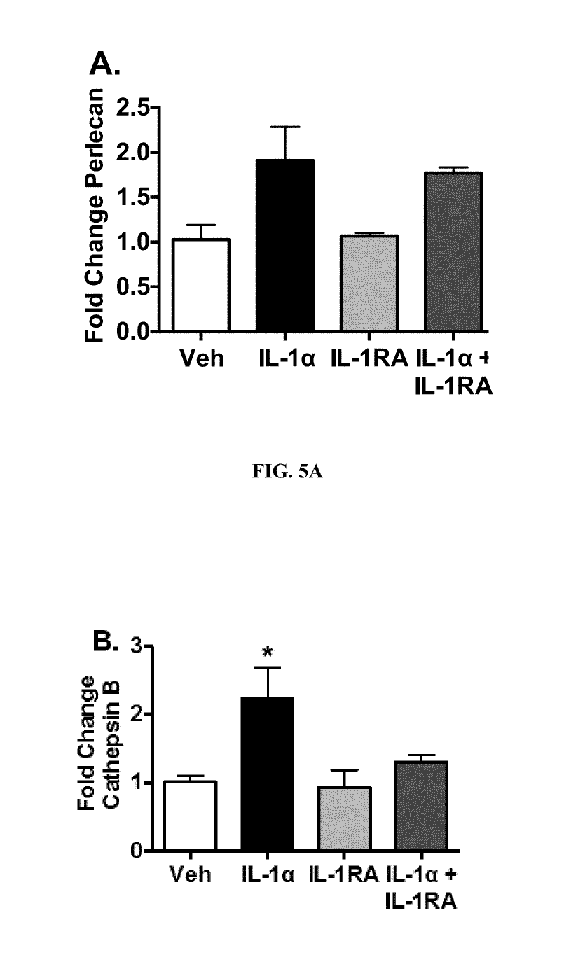

Taken together, this suggests IL-1α triggers neuroprotection and neurorepair directly on neurons, and indirectly, by inducing key neuroprotective and angiogenic factors. Our hypothesized IL-1α mechanisms of action are schematically summarized in FIG. 6.

These studies suggest the neuroprotective actions of IL-1α could be mediated by direct actions on neurons while at the same time inducing indirect multiple downstream mechanisms including the proteolytic cleavage of the vascular basement membrane (generation of the LG3 protein fragment from the protein core of perlecan) as well as expression of beneficial mediators such as PTX3 that can then induce generation of neuroprotective and angiogenic VEGF. For a long time, proteolysis of the vascular basement membrane was considered to be a marker of infarct expansion and poor outcomes. Previous studies have consistently demonstrated that this proteolysis may also be beneficial by its generation of neuroprotective and neuroreparative extracellular matrix fragments (42).

Methods: To determine the therapeutic potential of IL-1α in neuronal cultures treated with NMDA, primary cortical neurons from C57BL/6 mice were treated with NMDA (20 μM for 24 h) to induce excitotoxicity, and were co-treated acutely with IL-1α (0.1 or 10 ng/ml). Neuronal cell death was measured by lactate dehydrogenase (LDH) assay.

Results: We found that IL-1α triggered complete neuroprotection in primary neuronal cultures against NMDA-induced neurotoxicity (FIG. 4B). Additionally, we found that IL-1α mediated neuroprotection in primary neuronal cultures against OGD (FIG. 4A). Furthermore, we also show that PTX3 is highly neuroprotective against NMDA-induced neuronal cell death (FIG. 4C), and found that PTX3 stimulated the production of VEGF in neurons (FIG. 5C). VEGF has already been shown to be neuroprotective and proangiogenic (43).

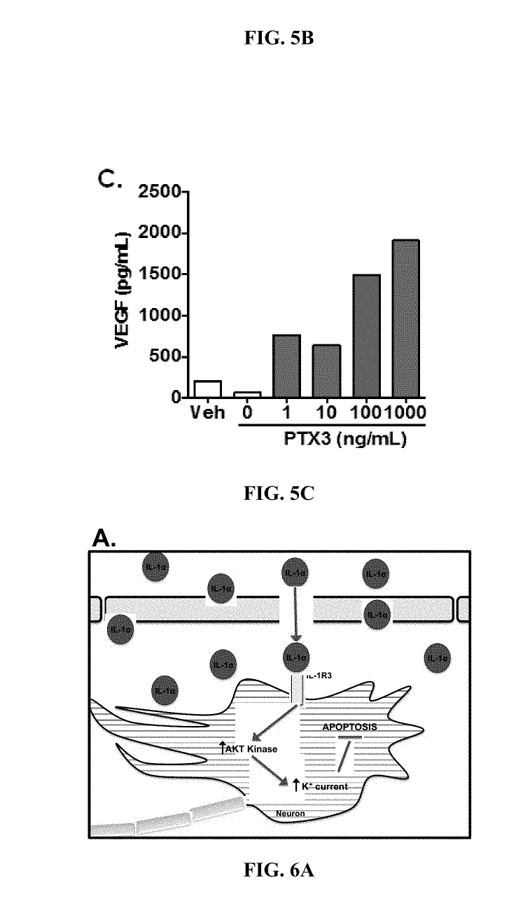

The relationship of IL-1α and perlecan on neuroprotection: In additional experiments, brain endothelial cells treated with IL-1α had increased mRNA levels of perlecan and cathepsin B (FIGS. 5A and 5B). Cathepsin B, in turn, cleaves perlecan to generate neuroprotective LG3. Perlecan hypomorph (Pln KO) mice, herein defined as expressing a truncated form of perlecan and also producing 10% of normal perlecan levels (but has no cerebrovascular phenotype), were investigated to determine response to IL-1α treatment (44). Pln KO mice treated with IV IL-1α immediately following stroke did not experience the same neuroprotection as did WT animals (FIG. 7), suggesting that IL-1α requires the C-terminal fragment of perlecan to exert its neuroprotective effect.

As discussed herein, systemic IV post-stroke administration of IL-1α resulted in changes in blood pressure (stabilized after stroke), heart rate (dropped slightly more than in stroked control animals). In additional studies, IV administration of IL-1α (0.1 or 1 ng) in naïve mice caused a slight transient increase in blood IL-6 levels that resolved after 24 hours (not shown). These changes in vital signs, while typical and expected of systemically administered IL-1α (35), are still undesirable for the potential clinical application of IL-1α in stroke, particularly in patients with co-existing comorbidity (infection, atherosclerosis). The stabilization of blood pressure could be beneficial through vasodilation and could also mediate neuroprotection. The decrease in heart rate, though transient and apparently non-life threatening, is one that should be avoided if possible. Indeed, dynamic cardiovascular changes (sudden alterations in heart rate and blood pressure) can be destabilizing in acute ischemic stroke. In the period prior to thrombolysis, decreases in heart rate and blood pressure may lead to reductions in cerebral perfusion, which, in the setting of an acute intracranial thrombus, may extend the core infarct. After thrombolysis (reopening the vessel by tPA and/or thrombectomy), rapid changes in heart rate and blood pressure may have a destabilizing effect, increasing the risk of hemorrhagic conversion of the stroke and complication.

An experimental model of acute selective intra-arterial (IA) drug administration after MCA recanalization was developed mo mimic clinical endovascular thrombectomy as described in the methods section of this example (36). Importantly, combining post-stroke IA thrombectomy with adjuvant neuroprotective therapy is highly clinically relevant since thrombectomy is now the standard of care for emergent large vessel occlusion; its use has increased 6-fold from 2004 to 2009 (37), but despite improving recanalization rates, there is often a disparity in clinical outcomes, with many patients not returning to independent function (36-70% post-thrombectomy) (37). This new approach of IA administration allows direct and therapeutic targeting of stroke-affected brain tissue with significantly smaller effective doses than the systemic IV dose (0.01 and 0.1 ng IA vs 1 ng IV), while simultaneously minimizing or eliminating systemic effects as suggested by preliminary results.

### Example 2

Based on the studies detailed in Example 1, it was anticipated that: 1. IL-1α administration will be well tolerated when administered in low systemic (IV) doses; 2. Animals treated with IL-1α IV will have less ischemic brain injury and better biological/histological (reduced signs of brain inflammation, BBB breakdown, and cellular apoptosis, etc.) and functional outcomes than stroked control mice; 3. IA administration of IL-1α immediately after recanalization will result in similar (or possibly better) therapeutic benefit but lesser or absent systemic side effects compared to IV administration; 4. Animals treated with combined acute and delayed IL-1α will have less damage and more effective neurorepair (angiogenesis and neurogenesis) than animals treated with acute regimen only; and 5. IL-1α administration will have age and/or gender differential effect. The following example continues the investigation of IL-1α and its neuroprotective effects.

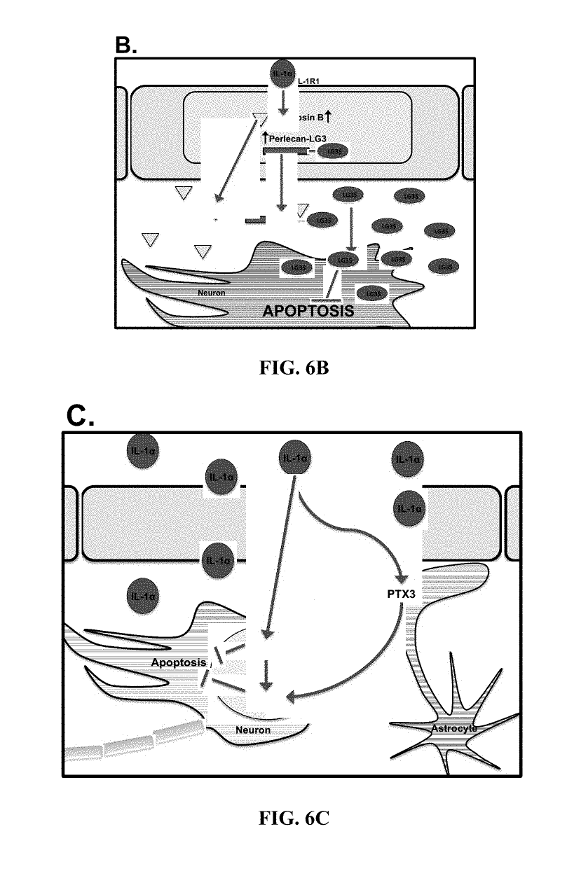

IL-1α is differentially upregulated in different cell types in various phases of stroke. IL-1α is expressed by many types of cells and this expression is known to be dependent on both disease state and time. Particularly, microglial, platelets, and astrocytes are known sources of IL-1α in various disease states. We were, therefore, interested to determine by which types of cells IL-1α was expressed following stroke. Current thinking is that the initial upregulation of IL-1α following ischemia occurs in as little as 4 hours, while, more recently it was shown that IL-1α is upregulated nearly 2 months post stroke. Our group was interested in seeing whether this persistent upregulation of IL-1α was through a single cell type or, as we hypothesized, its prolonged presence in the brain was due to several different types of parenchymal and peripheral cells. Due to the nature of IL-1α being an established pro-inflammatory cytokine, we decided to look at IL-1α expression changes in astrocytes and in microglia. Additionally, because of previously published work, we also chose to look at changes in IL-1α expression within platelets. Interestingly, we found that, at 24 hours post stroke, microglial IL-1α (FIG. 8A) accounted for nearly 80% of all IL-1α expression; while, at 14 days after stroke, it only accounted for 20% of all IL-1α expression within the periinfarct region (FIG. 8D). Inversely, at 24 hours after stroke, platelets accounted for none of the periinfarct IL-1α expression; while, after 2 weeks, platelets account for over 80% of the total IL-1α expression. Surprisingly, astrocytes accounted for none of the total IL-1α expression within the periinfarct region at either examined time point. Taken together, these data suggest that after the acute phases of stroke, the brain receives IL-1α from peripheral sources, thus making our treatment of stroke with IL-1α, less likely to significantly impact peripheral systems.

IL-1α is directly protective of primary cortical neurons after OGD in vitro. As a proof of concept, we first aimed to show that IL-1α could impart protection to neurons undergoing an in vitro stroke analogue such as oxygen glucose deprivation (OGD). Our original hypothesis was that IL-1α worked through another cell type (astrocytes or endothelia) to impart protection. To test this, primary cortical neurons from E18 mouse pups were plated on sterile coverslips and, after a week, underwent OGD or normoxia for 30 minutes. Reperfusion media was conditioned with either PBS vehicle or varying concentrations of IL-1α (0.01, 1, 10, and 100 ng/mL). After 24 hours reperfusion, we stained the cells with Hoechst nuclear stain for 30 minutes and then fixed and mounted. Cells were visualized on (microscope info here) and quantified for chromatin fragmentation and cellular health. Cells were classified as being healthy or unhealthy. We quantified 5 areas per coverslip or up to 200 healthy cells with 9 coverslips per treatment group. We found that IL-1α significantly increased the cell viability after OGD. Interestingly, although not surprisingly, the lowest and highest doses were not beneficial and, in fact, the highest doses were detrimental even under normoxic conditions. What was surprising, and against our original hypothesis, was that IL-1α was directly protective of a relatively fragile cell type. This supports the idea that IL-1α, being a cytokine, is still a viable therapeutic option under appropriate dosing regimens. N=3.

IL-1α treatment is safe up to 200,000× its effective dose. It has long been established that IL-1α is an early mediator of fever and an early signaling molecule in sepsis. Our published in vitro research, as well as the study above, demonstrated that, at low doses, we can induce reparative processes in endothelial cells and direct protection of primary neurons. In this experiment, we wanted to identify the dose at which IL-1α becomes unsafe in mice. We defined this threshold based on previous findings defining fever in a mouse to be 1 degree C. sustained increase in body temperature. We went on to classify that increase as a “mild” fever and classified 2.5 or more degrees Celsius sustained increase in body temperature as being a severe fever. In animals that underwent MCAo surgery, we administered 5, 7.5 and 10 mg/kg of IL-1α via tail vein injection and monitored core body temperature by rectal probe (along with other vital statistics such as heart rate and pulse distension which is analogous to blood pressure in mice). None of the mice who received 5 mg/kg developed fever. 50% of the mice who received 7.5 mg/kg developed fever with at least 1 of them developing severe fever. Finally, 75% of the mice receiving 10 mg/kg developed fever with all of them having classified as sustained severe fever. This suggests even if we increased our chosen dose 10⁵-fold, it would still be safe to administer without the mouse sustaining significant side effects.

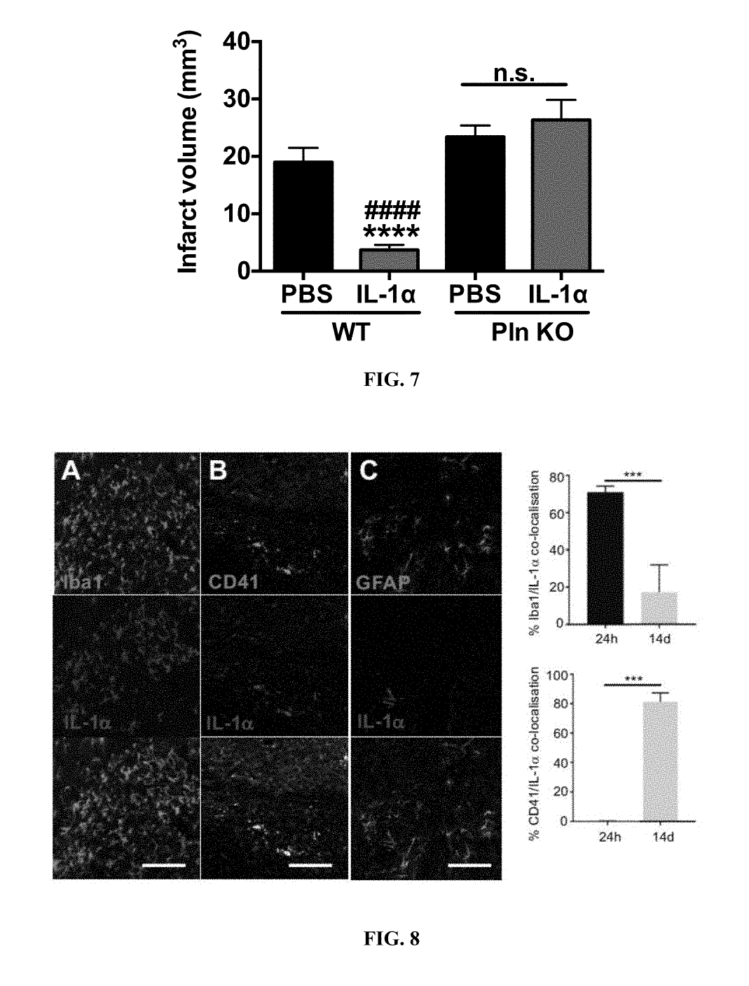

IL-1α reduces infarct volume and apoptotic cell death following stroke. In order to show that IL-1α could be an attractive therapeutic target following stroke, we first wanted to demonstrate its efficacy at preventing ischemic injury. We also wanted to show that IL-1α was an attractive candidate for IA drug delivery using our model (Maniskas et al, 2015). At 7 days after stroke, animals which received IL-1α showed significantly lower levels of apoptotic cell death on TUNEL staining (FIGS. 9A and B) as well as lower overall infarct volumes on cresyl violet staining (FIG. 9C). Interestingly, while IA IL-1α administration did not lessen overall infarct volumes compared with IV IL-1α administration, IA IL-1α appears to decrease apoptotic cell death compared to IV IL-1α although this effect is not statistically significant (p=0.1708).

IL-1α reduces intraparenchymal inflammatory activation after stroke. We next wanted to see if IL-1α, being a pro-inflammatory cytokine, instigated widespread inflammatory activation within the brain. Unsurprisingly, we saw that stroked animals had widespread microglial (CD11b) and astrocyte (GFAP) activation. What did surprise us was that animals receiving IV IL-1α showed decreased GFAP and CD11b staining compared to control. Additionally, animals which received IA IL-1α showed even less GFAP and CD11b staining compared to IV IL-1α. These data suggest that, contrary to conventional wisdom, the administration of low dose IL-1α could surprising decrease the inflammatory response to stroke.

IL-1α improves functional outcomes following stroke. In order to solidify IL-1α as an attractive post stroke therapeutic target, we aimed to show that these IL-1α treated animals received functional benefit compared to their PBS treated counterparts upon behavioral testing. Mice underwent a battery of behavioral tests (which we compiled into an 11-point neurological score) and open field testing. While there were no significant differences in our compiled behavioral score, we found a few notable differences during the open field trials. Upon open field testing, we found that both IV and IA treated animals traveled farther in overall distance than their control animal counterparts. Additionally, animals in both IV and IA treated groups spent more time in the open areas of the arena rather than staying near the walls. This suggests that these animals are both more mobile and are not exhibiting elevated anxiety compared to controls. Even more interestingly, these effects become more pronounced with increased time after stroke. Taken together, we found that, regardless of treatment modality, acute IL-1α treatment improves functional outcomes after stroke.

Perlecan plays an important role in IL-1α mediated neuroprotection after stroke. Finally, we wanted to determine the mechanism by which IL-1α could be working. Our past work suggested that elements of the extracellular matrix, such as perlecan, are broken down after stroke and that this process could partially be driven by IL-1α (Saini et al 2011). Additionally, we know that one of these proteolytic fragments, perlecan LG3, is neuroprotective following OGD. In order to see whether this protection is translated in mice, we used a mouse with truncated perlecan, that is, perlecan lacking the LG3 fragment (pln −/−). Interestingly, while WT mice exhibited neuroprotection following IL-1α treatment, pln −/− mice did not gain the same neuroprotection after IL-1α treatment. This shows that IL-1α works through the generation of LG3 from full length perlecan.

### Materials and Methods

IL-1α protein preparation: Upon arrival, mouse recombinant IL-1α and IL-1β (R&D Systems, Minneapolis, Minn., USA) were diluted in sterile phosphate-buffered saline containing 0.1% low endotoxin bovine serum albumin (also used as vehicle control). To avoid freeze thaw cycles, the diluted stock solution (50 μg/mL) was then aliqotted and frozen for dilution to the desired dose on the day of surgery.

### Surgical Methods

Tandem Ipsilateral Common Carotid and Middle Cerebral Artery Occlusion Stroke Model: The experimental protocol was approved by the Institutional Animal Care and Use Committee of the University of Kentucky and experiments were performed in accordance with the Guide for the Care and Use of Laboratory Animals of the National Institutes of Health as well as the ARRIVE guidelines. Male (3 months old) C57/B16 mice were subjected to transient tandem ipsilateral common carotid artery (CCA)/middle cerebral artery (MCA) occlusion (MCAo) for 60 min (Lee et al. 2011), followed by reperfusion of both arteries for up to 7 days. A small burr hole was made in the skull to expose the MCA and a metal wire with a diameter of 0.005 inch was placed under the artery. Slight elevation of the metal wire causes visible occlusion of the MCA. The CCA was then isolated and occluded using an aneurysm clip. Diminished blood flow was confirmed with Laser Doppler Perfusion Monitor (Perimed, USA) and only those animals with a diminished blood flow of at least 80% and re-establishment of at least 75% of baseline levels were included in subsequent experimentation.

Middle Cerebral Artery Occlusion Model: 3-month-old mice underwent middle cerebral artery occlusion as previously described (ref). Briefly, mice were anesthetized and their external carotid artery isolated and permanently ligated distal to its bifurcation from the common carotid artery. A filament was then inserted and advanced within the internal carotid artery to its bifurcation into middle cerebral artery. The filament was then anchored and allowed to remain there for 60 minutes. The filament was then removed and the ECA permanently ligated constituting recanalization.

Intraarterial drug administration of IL-1α: Animals in the IA drug delivery cohort underwent IA drug deliver as previously described (Maniskas et al 2015). Briefly, the mouse was placed in a supine position with the previously isolated CCA exposed. Following the CCA rostrally to its bifurcation point, the internal and external carotid arteries (ICA and ECA respectively) were identified and three lengths of 6-0 suture were placed under the ECA, ensuring its isolation. In order to create a closed system to minimize blood loss, one of the sutures was used to ligate the ECA distally to the bifurcation while a microclamp was placed on the ICA. The ECA was then nicked just proximally to the ligation point and the drug delivery tubing was inserted into the nicked vessel. A suture was used to secure the tubing for the duration of drug delivery. Once the tubing was successfully placed, the mouse underwent the reperfusion phase of the tandem ipsilateral common carotid and middle cerebral artery occlusion stroke model (as described above), the clamp on the ICA was removed, and 10-25 uL drug was administered at a rate of 10 uL per minute. Following drug administration, a suture was used to ligate the ECA proximal to the nick and the tubing was removed. The mouse was then allowed to recover for the duration of the study (3 to 7 days).

Treatment with IL-1α: Each mouse received 0.05 μg/kg IL-1α (approximately 1 ng per 100 μL of PBS) via tail vein (IV) injection or 0.005 μg/kg via IA injection. Injections were performed on anesthetized adult mice immediately following recanalization of occluded vessels. All mice were treated on the day of surgery and allowed to recover until PSD 3 or 7.

### Behavioral Assessments

11-point Behavioral Neurological Score: Mice underwent behavioral assessment to assess the following behavioral metrics: level of consciousness (LOC), gaze (G), visual field (VF), sensorimotor response (SR), and grip strength and endurance/ paralysis paw hang (PPH). LOC was determined prior to any disturbance of the animal's cage and was assessed on a 0-2 severity scale with 0 being alert and active without outside stimulus, 1 being responsive to stimulus, and 2 being huddled, unresponsive, and non-grooming. Gaze was assessed by passing a visual stimulus in front of each eye in turn WITHOUT disturbing the mouse's whiskers. The subject was given a 0 score if they looked toward the stimulus and a 1 if they failed to do so. VF was assessed by holding the mouse by the tail near a platform (on its right or left side) and if the mouse reached for the platform it received a score of 0. If it did not reach within 5 seconds, it was given a score of 1 for each side it failed on. SR was scored by pressing each paw in turn to elicit a reaction. A reaction was defined as: vocalizing pain, retracting the paw, or jumping in response to the paw press. A lack of any of these signs resulted in a score of 1 for each paw affected. Finally, PPH was scored by a typical paw hang test. The mouse uses its front paws to hang from a rod for a period of 60 seconds. The mouse receives a score of 0 if it is able to hang with both paws without dropping a paw below the level of the rod for the full 60 seconds. A score of 1 is earned if the mouse drops either paw without falling. The time of the first “partial” paw drop is also recorded. A score of 2 is earned if the mouse falls, releasing both paws, at any time during the 60 second time period. The total scores are tallied at the conclusion of the testing to assess overall function. Other summary metrics such as “latency to first paw drop” were also used to help assess fine motor function.

Open Field Behavioral Assessment: Each subject was placed in its own 2×2 box and tracked using the EthoVision 12 software for 5 minutes. Animals were assessed on the day prior to stroke surgery, and then again on PSD 1, 3, and 7. Parameters tracked include total distance traveled, average velocity, turn angle, and time spent in center zone. The center zone was defined as being all area within the box that was at least 5 inches away from the walls of the box. This parameter allowed us to track anxiety as a function of how long the animal ventured into the center of the box.

### Histology

Morphological Stains: Gross cellular morphology was assessed using Cresyl Violet staining. Mounted sections were fixed with 10% phosphate buffered formalin. They were then stained using standard Cresyl Violet staining methods, mounted using DPX Mountant medium, and were scanned using a HP Scanjet G4050. The scanned images were analyzed using NIH Image J for infarct volume measurement (Lee et al., 2011). Infarct regions were defined as regions with hypodense cresyl violet staining reflecting areas of dead or dying nuclei. Areas were calculated using the ImageJ free-hand selection tool and summated to calculate final infarct volume.

TTC staining: Upon removal, the brain was placed in a 1 mm matrix and was cut into 2 mm coronal sections. The sections were submerged in a 1% 2,3,5 Triphenyltetrazolium chloride (TTC) solution for 15 minutes. They were then removed from solution and were scanned using a HP Scanjet G4050. Normal, healthy tissue turns red upon TTC's interaction with healthy mitochondria. Dead and dysfunctional mitochondria remain white thus highlighting the infarcted region. Areas were calculated using the ImageJ free-hand selection tool and summated to calculate final infarct volume.

Immunohistochemistry: Mounted, 20 μm tissue sections were fixed with ice cold 1:1 acetone:methanol prior to incubating in blocking buffer (5% BSA in PBS with 0.1% Triton X-100) for one hour at room temperature. The sections were then incubated overnight at 4° C. in primary antibody (in 2% BSA/0.1% Triton X-100) against Ki67, CD11b, GFAP, and NeuN. Sections were washed and incubated with a fluorescent secondary antibody (1:1000; AlexaFluor 488 or 568, Life Technologies) for one hour at room temperature. Sections were washed again and then coverslipped with fluorescent mounting media containing DAPI (H-1200, Vector Labs) and images were captured using a Nikon Eclipse Ti microscope and software (Nikon). Images were analyzed for antibody-specific positive staining using ImageJ (threshold pixel intensity made similar across all images to isolate antibody-specific staining and then recorded the number of stain positive pixels). Results are from 3 sections per animal and the area selected was in the infarct core identified morphologically.

Statistical Analysis: All experiments were performed in duplicated studies, and each treatment group contained at least 4 mice. Data are represented as mean±SE of the mean (SEM). Comparison between two groups was done using the Student's t-test. Comparison between three or more groups was performed using one-way ANOVA followed by a Tukey's post hoc analysis. For CXCL1 and IL-6 ELISA assays, data were analyzed by two-way ANOVA and Bonferroni post hoc test. Significance was determined by a p value of <0.05.

### Discussion

We set out to show that IL-1α is an attractive therapeutic target for post stroke intervention. Our previous research described that IL-1α's overall expression in the brain remained elevated up to a week after stroke (Salmeron et al 2015). In order to more clearly determine IL-1α's pattern of expression, we tested cells that have been recognized in the art to be IL-1α contributors in various disease states. We were unsurprised to find that microglia were the principal cellular source of IL-1α acutely and that platelets are the principle source of IL-1α in more chronic phases of stroke injury. We were surprised, however, to find that astrocytes did not contribute to IL-1α production at 1 day and 14 days after stroke. It is possible that astrocytic contribution of IL-1α expression occurs at an interim time point which we did not investigate. However, other labs have more recently discovered that IL-1α is elevated even out to 7 weeks following ischemic injury. This finding suggests that IL-1α could have additional, significant roles in more long-term phases of injury progression and repair and clearly highlights a new area for future investigation.

Stuart Allan's group in Manchester, England has definitively shown that IL-1α is one of the first cytokines upregulated after stroke (Luheshi et al, 2011). We recently showed that IL-1α treatment of endothelial cells showed benefit in vitro (Salmeron et al). We, therefore, reasoned that a cytokine, present so early in stroke injury progression, could be a prominent player in infarct evolution and could even be beneficial to cortical neurons. We found that under OGD conditions IL-1α imparted neuroprotection onto primary cortical neurons. More interesting, was the fact that IL-1α was protective only under certain doses. The highest doses and lower doses were not significantly protective and, in the case of the highest dose of 100ng/mL, the IL-1α was neurotoxic. This told us that we would need to find which doses of IL-1α treatment which would be detrimental (or even lethal) in vivo and compare that dose to the doses that have been used by other groups as well as by our own group.

In order to confirm that the previously established dose of ˜0.05 μg/kg was safe, we originally set out to find IL-1α's LD₅₀ in mice. Interestingly, our highest dose, 10 mg/kg, which was 10⁵ times greater than the established dose was not lethal. This shifted our focus to seeing whether any of the doses tested resulted in negative side effects. IL-1α is a known mediator of fever and so we chose fever as our warning symptom. As described above, we found that 7.5 mg/kg produced 50% fever out to at least 30 minutes following recanalization. While none of these mice died during or after the injections, these results clearly show that the IL-1α that we injected was both active and could, at high enough doses, produce severe fever as well as other hemodynamic changes such as blood pressure and heart rate. These symptoms are dangerous enough to exclude it as a potential therapy. It was therefore important to us to verify these “danger” thresholds in our studies to both show that our chosen therapeutic dose was safe and to show just how far removed our dose is compared to those doses which produce negative side effects.

After showing that our established dose was safe, we proceeded to show that treating mice with IL-1α is neuroprotective. We showed that even though our dose does not produce severe fever or major complications, systemic administration (IV injection) of IL-1α does result in transient, mild hemodynamic changes such as fleeting changes in pulse distension (analogous to blood pressure) and heart rate. Our intra-arterial model of drug delivery gave us the opportunity to administer less IL-1α, thereby lessening the chance of inducing the side effects outlined above. It also allowed us to deliver the IL-1α nearly directly to the location of the stroke. Additionally, since it is known that IL-1α is transported across the blood-brain barrier (BBB), we were able to be reasonably confident that most of our drug was taken up into the brain parenchyma rather than being metabolized in systemic circulation. While we know that IL-1α is transported and we are confident that our drug is taken up into the brain, this is an area which is actively being pursued in the lab.

Perhaps most importantly, IL-1α facilitates functional recovery and decreases neuroinflammatory activation particularly when it is delivered IA. There are a few potential reasons for this reduction in neuroinflammation. First, we are giving less drug and so any potential inflammatory activation caused by the introduction of a cytokine is reduced compared to our IV dose. Second, IL-1α could be working through another mechanism of neuroprotection thereby reducing the inflammatory response secondary to smaller overall injury. Finally, IL-1α could be working through a negative feedback loop to reduce inflammatory cell activation. This could be in part through its interaction with the BBB and perlecan's generation of LG3. Naturally, a combination of these reasons is likely plausible, however, more studies will need to be done in order to parse out which potential reason for the observed reduction in inflammation is most correct.

Finally, we were able to use IL-1α and our recently developed model as a proof of concept for giving potentially life-saving drugs using a safer drug delivery mechanism. Our stroke model produces infarcts that are similar to those resulting from large vessel occlusions in the clinic. Endovascular thrombectomy gives clinicians a great opportunity to deliver drugs in a targeted fashion immediately following vessel recanalization. Our IA drug delivery method models this targeted drug delivery very closely. This is a chance for drugs to be reexamined as post stroke therapeutics that have been previously discarded on the basis of producing side effects.

Taken together, our studies have shown that IL-1α is an attractive therapeutic target for neuroprotective intervention after experimental ischemic stroke. We have shown that IL-1α is directly neuroprotective in vitro and that it is neuroprotective when given IV or IA (with IA delivery imparting even greater benefit) potentially through perlecan releasing LG3. We believe that IL-1α combined with endovascular thrombectomy could represent a new avenue for stroke treatment for this new era of post stroke treatment.

### Example 3

The present study extends our previous findings and demonstrate for the first time that exogenous intravenous administration of subpathological doses of IL-1α is highly neuroprotective when injected during the acute phase of injury, and enhances post-stroke angiogenesis when injected during the sub-acute phase of ischemic injury. These results have very important and surprising implications by proposing for the first time that complete inhibition of post-stroke neuroinflammation may have detrimental effects, whilst sustaining low grade chronic inflammation might be used as new effective therapy for brain tissue repair and functional recovery after stroke.

### Results

IL-1α treated animals have less (subacute) tissue damage than vehicle. First, we aimed to discern IL-1α's ability to affect overall tissue morphology when given following stroke. In this experiment, we treated stroked animals via tail vein injection at post stroke day (PSD 3) in an effort to avoid affecting neuroprotective pathways. As our model typically reaches its maximum volume at day 3, we chose that day to treat our mice. Upon sacrifice, brains were analyzed for overall tissue morphology using H&E staining as described above. We found that animals treated with IL-1α had less overall tissue dysmorphia than their vehicle control counterparts.

IL- 1 a treated animals exhibit accelerated functional recovery after delayed IL-1α treatment. We next wanted to determine whether IL-1α treatment enhances functional benefit when given 3 days following stroke. The animals described above were subjected to a battery of neurological tests summated into an 11-point neurological score. Both groups showed similar functional deficit on the day following stroke surgery as expected. 1 day after treatment, at PSD4, the animals treated with IL-1α showed significant functional recovery compared to vehicle controls. This effect seemed to diminish as the mice recovered suggesting the potential need for a multi dose treatment regimen instead.

IL-1α treated animals have more activated endothelial cells in the periinfarct region. Next, we looked to see whether our overall histological findings as well as the observed functional benefit could be due to a “jump started” angiogenic response. Tissue sections were therefore stained for ICAM-1, a known marker for endothelial cell activation. We found that animals treated with IL-1α had significantly more ICAM-1 positive staining than did animals receiving vehicle treatment. We observed that while all histological data came from the periinfarct areas, ICAM-1 expression was only particularly strong in the striatal periinfarct.

IL-1α treated animals have more vascular density in the periinfarct region. Because of the observed striatal endothelial cell activation, we next looked to see whether there was any evidence that cortical angiogenesis had already concluded. To do this, we stained for overall vascular density using PECAM-1 (CD31). We found that IL-1α treated animals had increased overall vascular density in all periinfarct regions (including in the striatum). This suggested to us that we had missed the window for cortical angiogenesis and that we were catching the end of striatal angiogenesis.

### Materials and Methods

Recombinant IL-1α protein preparation: Mouse recombinant IL-1α and IL-1β (R&D Systems, Minneapolis, Minn., USA) were diluted in sterile phosphate-buffered saline containing 0.1% low endotoxin bovine serum albumin (also used as vehicle control). To avoid freeze thaw cycles, the diluted stock solution (50 μg/mL) was then aliquotted and frozen for dilution to the desired dose on the day of surgery.

### Surgical Methods

Experimental protocols were approved by the Institutional Animal Care and Use Committee of the University of Kentucky, as well as the Home Office (UK), and experiments were performed in accordance with the Guide for the Care and Use of Laboratory Animals of the National Institutes of Health as well as the ARRIVE guidelines

Tandem ipsilateral common carotid and middle cerebral artery occlusion stroke model. Male (3 months old) C57/B16 mice were subjected to transient tandem ipsilateral common carotid artery (CCA)/middle cerebral artery (MCA) occlusion (MCAo) for 60 min (Lee et al. 2011), followed by reperfusion of both arteries for up to 7 days. A small burr hole was made in the skull to expose the MCA and a metal wire with a diameter of 0.005 inch was placed under the artery. Slight elevation of the metal wire causes visible occlusion of the MCA. The CCA was then isolated and occluded using an aneurysm clip. Diminished blood flow was confirmed with Laser Doppler Perfusion Monitor (Perimed, USA) and only those animals with a diminished blood flow of at least 80% and re-establishment of at least 75% of baseline levels were included in subsequent experimentation.

Middle Cerebral Artery Occlusion Model: 3-month-old mice underwent middle cerebral artery occlusion (MCAo) as previously described protocol (REF). Briefly, a hole was made into the temporalis muscle (6 mm lateral and 2 mm posterior from bregma) to allow a 0.5 mm diameter flexible laser-Doppler probe to be fixed onto the skull and secured in place by tissue adhesive (Vetbond). A midline incision was made on the ventral surface of the neck and the right common carotid artery isolated and ligated. Topical anaesthetic (EMLA, 5% prilocaine and lidocaine, AstraZeneca, UK) was applied to skin incision sites prior to incision. The internal carotid artery and the pterygopalatine artery were temporarily ligated. A 6-0 monofilament (Doccol, Sharon, Mass., USA) was introduced into the internal carotid artery via an incision in the common carotid artery. The filament was advanced approximately 10 mm distal to the carotid bifurcation, beyond the origin of the middle cerebral artery. Relative CBF was monitored for the first 30-45 min following MCAo, during which time relative CBF had to reduce by at least 70% of pre-ischemic values for inclusion. After 30 min of occlusion, the filament was withdrawn back into the common carotid artery to allow reperfusion to take place. The wound was sutured and mice received a subcutaneous bolus dose of saline for hydration (500 μl) and a general analgesic (Buprenorphin e, 0.05 mg/kg injected subcutaneously). Animals were kept at 26-28° C. until they recovered from anaesthesia and surgery, before being transferred back to ventilated cages suspended over a heating pad with free access to mashed food and water in normal housing conditions.

Treatment with IL-1α: Each mouse received 0.05 mg/kg IL-1α (approximately 1 ng per 100 uL of PBS) via tail vein (IV) injection. Injections were performed on un-anesthetized adult mice using a mouse restrainer to avoid confounding affects with anesthesia. For single injection studies, all mice were treated on post stroke day (PSD) 3 and allowed to recover until PSD 14. For multiple injection studies, mice were treated on PSD 3, 6, and 9 and again allowed to recover until PSD 14.

### Behavioral Assessments

28-point Neurological Assessment: Mice underwent baseline behavioral assessment on the day prior to stroke.

11-point Behavioral Neurological Score: Mice underwent behavioral assessment to assess the following behavioral metrics: level of consciousness (LOC), gaze (G), visual field (VF), sensorimotor response (SR), and grip strength and endurance/ paralysis paw hang (PPH). LOC was determined prior to any disturbance of the animal's cage and was assessed on a 0-2 severity scale with 0 being alert and active without outside stimulus, 1 being responsive to stimulus, and 2 being huddled, unresponsive, and non-grooming. Gaze was assessed by passing a visual stimulus in front of each eye in turn WITHOUT disturbing the mouse's whiskers. The subject was given a 0 score if they looked toward the stimulus and a 1 if they failed to do so. VF was assessed by holding the mouse by the tail near a platform (on its right or left side) and if the mouse reached for the platform it received a score of 0. If it did not reach within 5 seconds, it was given a score of 1 for each side it failed on. SR was scored by pressing each paw in turn to elicit a reaction. A reaction was defined as: vocalizing pain, retracting the paw, or jumping in response to the paw press. A lack of any of these signs resulted in a score of 1 for each paw affected. Finally, PPH was scored by a typical paw hang test. The mouse uses its front paws to hang from a rod for a period of 60 seconds. The mouse receives a score of 0 if it is able to hang with both paws without dropping a paw below the level of the rod for the full 60 seconds. A score of 1 is earned if the mouse drops either paw without falling. The time of the first “partial” paw drop is also recorded. A score of 2 is earned if the mouse falls, releasing both paws, at any time during the 60 second time period. The total scores are tallied at the conclusion of the testing to assess overall function. Other summary metrics such as “latency to first paw drop” were also used to help assess fine motor function.

Open Field Behavioral Assessment: Each subject was placed in its own 2×2 box and tracked using the EthoVision 12 software for 5 minutes. Animals were assessed on the day prior to stroke surgery, and then again on PSD 1, 3, and 7. Parameters tracked include total distance traveled, average velocity, turn angle, and time spent in center zone. The center zone was defined as being all area within the box that was at least 5 inches away from the walls of the box. This parameter allowed us to track anxiety as a function of how long the animal ventured into the center of the box.

### Histology

Morphological Stains: Gross cellular morphology was assessed using Hematoxylin Eosin staining. Mounted sections were fixed with 10% phosphate buffered formalin. They were then stained using standard H&E methods with Gill's Hematoxylin, mounted using DPX Mountant medium, and were scanned using a HP Scanj et G4050. The scanned images were input into NIH Image J for infarct analysis (Lee et al., 2011). H&E dismorphic areas were defined as regions with loss, lower density, smaller, irregular shaped nuclei or irregular tissue patterning from surrounding areas. Areas were calculated using the ImageJ free-hand selection tool and summated to calculate final dysmorphic volume.

Immunohistochemistry: Mounted, 20 um tissue sections were fixed with ice cold 1:1 acetone:methanol prior to incubating in blocking buffer (5% BSA in PBS with 0.1% Triton X-100) for one hour at room temperature. The sections were then incubated overnight at 4° C. in primary antibody (in 2% BSA/0.1% Triton X-100) against PECAM (1:100) Sections were washed and incubated with a fluorescent secondary antibody (1:1000; AlexaFluor 488 or 568, Life Technologies) for one hour at room temperature. Sections were washed again and then coverslipped with fluorescent mounting media containing DAPI (H-1200, Vector Labs) and images were captured using a Nikon Eclipse Ti microscope and software (Nikon). Images were analyzed for antibody-specific positive staining using ImageJ (threshold pixel intensity made similar across all images to isolate antibody-specific staining and then recorded the number of stain positive pixels). Results are from 3 sections per animal and the area selected was in the infarct core identified morphologically.

Statistical Analysis: All experiments were performed on at least replicated studies, and each treatment group contained at least 4 mice. Data are represented as mean±SE of the mean (SEM). Comparison between two groups was done using the Student's t-test. Comparison between three or more groups was performed using one-way ANOVA followed by a Tukey's post hoc analysis. We analyzed time course behavioral data (such as foot fault and 11-point neuroscore) by two-way ANOVA and Bonferroni post hoc test. Significance was determined by a p value of <0.05.

While the terms used herein are believed to be well understood by one of ordinary skill in the art, definitions are set forth herein to facilitate explanation of the presently-disclosed subject matter.

Unless defined otherwise, all technical and scientific terms used herein have the same meaning as commonly understood by one of ordinary skill in the art to which the presently-disclosed subject matter belongs. Although any methods, devices, and materials similar or equivalent to those described herein can be used in the practice or testing of the presently-disclosed subject matter, representative methods, devices, and materials are now described.

Following long-standing patent law convention, the terms “a”, “an”, and “the” refer to “one or more” when used in this application, including the claims. Thus, for example, reference to “a protein” includes a plurality of such proteins, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments±20%, in some embodiments±10%, in some embodiments±5%, in some embodiments±1%, in some embodiments±0.5%, and in some embodiments±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

Throughout this document, various references are mentioned. All such references are incorporated herein by reference.

### REFERENCES

1. Pradillo J M, Denes A, Greenhalgh A D, Boutin H, Drake C, McColl B W, Barton E, Proctor S D, Russell J C, Rothwell N J, Allan S M. Delayed administration of interleukin-1 receptor antagonist reduces ischemic brain damage and inflammation in comorbid rats. J Cereb Blood Flow Metab. 2012;32(9):1810-9. doi: 10.1038/jcbfm.2012.101. PubMed PMID: 22781338; PMCID: PMC3434631.

2. Clausen B H, Lambertsen K L, Dagnaes-Hansen F, Babcock A A, von Linstow C U, Meldgaard M, Kristensen B W, Deierborg T, Finsen B. Cell therapy centered on IL-1Ra is neuroprotective in experimental stroke. Acta Neuropathol. 2016;131(5):775-91. doi: 10.1007/s00401-016-1541-5. PubMed PMID: 26860727; PMCID: PMC4835531.

3. Emsley H C, Smith C J, Georgiou R F, Vail A, Hopkins S J, Rothwell N J, Tyrrell P J, Acute Stroke I. A randomised phase II study of interleukin-1 receptor antagonist in acute stroke patients. J Neurol Neurosurg Psychiatry. 2005;76(10):1366-72. doi: 10.1136/jnnp.2004.054882. PubMed PMID: 16170078; PMCID: PMC1739363.

4. Luheshi N M, Kovacs K J, Lopez-Castejon G, Brough D, Denes A. Interleukin-lalpha expression precedes IL-1beta after ischemic brain injury and is localised to areas of focal neuronal loss and penumbral tissues. J Neuroinflammation. 2011;8:186. doi: 10.1186/1742-2094-8-186. PubMed PMID: 22206506; PMCID: PMC3259068.

5. Um J. Interleukin-1 gene cluster polymorphisms in cerebral infarction. Cytokine. 2003;23(1-2):41-6. doi: 10.1016/s1043-4666(03)00183-2.

6. Um J-Y, Moon K-S, Lee K-M, Yun J-M, Cho K-H, Moon B-S, Kim H-M. Association of interleukin-1 alpha gene polymorphism with cerebral infarction. Molecular Brain Research. 2003;115(1):50-4. doi: 10.1016/50169-328x(03)00179-7.

7. Rodriguez-Grande B, Swana M, Nguyen L, Englezou P, Maysami S, Allan S M, Rothwell N J, Garlanda C, Denes A, Pinteaux E. The acute-phase protein PTX3 is an essential mediator of glial scar formation and resolution of brain edema after ischemic injury. J Cereb Blood Flow Metab. 2014;34(3):480-8. doi: 10.1038/jcbfm.2013.224. PubMed PMID: 24346689; PMCID: PMC3948128.

8. Rodriguez-Grande B, Varghese L, Molina-Holgado F, Rajkovic O, Garlanda C, Denes A, Pinteaux E. Pentraxin 3 mediates neurogenesis and angiogenesis after cerebral ischaemia. J Neuroinflammation. 2015;12:15. doi: 10.1186/s12974-014-0227-y. PubMed PMID: 25616391; PMCID: PMC4308938.

9. Salmeron K, Redondo-Castro E, Aihara T, Pinteaux E, Bix G J. IL-1alpha induces angiogenesis in brain endothelial cells in vitro: implications for brain angiogenesis after acute injury. J Neurochem. 2015.

10. Saini M G, Pinteaux E, Lee B, Bix G J. Oxygen-glucose deprivation and interleukin-1alpha trigger the release of perlecan LG3 by cells of neurovascular unit J. Neurochem 011; 119(4):760-71, doi:10.1111/j.1471-4159.2011.07844.x.

11. White E S, Mantovani A R. Inflammation, wound repair, and fibrosis: reassessing the spectrum of tissue injury and resolution. J Pathol. 2013;229(2):141-4. doi: 10.1002/path.4126. PubMed PMID: 23097196; PMCID: PMC3996448.

12. Qian J, Zhu L, Li Q, Belevych N, Chen Q, Zhao F, Herness S, Quan N. Interleukin-1R3 mediates interleukin-1-induced potassium current increase through fast activation of Akt kinase. PNAS. 2012; 109(30): 12189-94.

13. Chaskiel L, Konsman JP. Neuroimmune Biology. In: Phelps C, Korneva E, Berczi I, Szentivanyi A, editors. Cytokines and the Brain. Amsterdam: Elsevier; 2008.

14. Cheng W, Shivshankar P, Zhong Y, Chen D, Li Z, Zhong G. Intracellular interleukin-1alpha mediates interleukin-8 production induced by Chlamydia trachomatis infection via a mechanism independent of type I interleukin-1 receptor. Infect Immun. 2008;76(3):942-51. doi: 10.1128/IAI.01313-07. PubMed PMID: 18086816; PMCID: PMC2258806.

15. Thornton P, McColl B W, Greenhalgh A, Denes A, Allan S M, Rothwell N J. Platelet interleukin-lalpha drives cerebrovascular inflammation. Blood. 2010;115(17):3632-9. doi: 10.1182/blood-2009-11-252643. PubMed PMID: 20200351.

16. Hawrylowicz C M, Santoro S A, Platt F M, Unanue E R. Activated platelets express IL-1 activity. J Immunol. 1989;143(12):4015-8.

17. Burrows F, Haley M J, Scott E, Coutts G, Lawrence C B, Allan S M, Schiessl I. Systemic inflammation affects reperfusion following transient cerebral ischemia. Exp Neurol. 2016;277:252-60.

18. Rivera F J, Kazanis I, Ghevaert C, Aigner L. Beyond Clotting: A Role of Platelets in CNS Repair? Front Cell Neurosci. 2015;9:511. doi: 10.3389/fnce1.2015.00511. PubMed PMID: 26834562; PMCID: PMC4718976.

19. Hayon Y, Dashevsky O, Shai E, Varon D, Leker R R. Platelet lysates stimulate angiogenesis, neurogenesis and neuroprotection after stroke. Thrombosis and Haemostasis. 2013;110(2):323-30.

20. Franceschi C, Campisi J. Chronic inflammation (inflammaging) and its potential contribution to age-associated diseases. J Gerontol A Biol Sci Med Sci. 2014;69 Suppl 1:S4-9. doi: 10.1093/gerona/g1u057. PubMed PMID: 24833586.

21. Genazzani A R, Pluchino N, Luisi S, Luisi M. Estrogen, cognition and female ageing. Hum Reprod Update. 2007;13(2):175-87. doi: 10.1093/humupd/dm1042. PubMed PMID: 17135285.

22. Hacham M, Argov S, White R, Segal S, Apte R. Different patterns of interleukin-lalpha and interleukin-lbeta expression in organs of normal young and old mice. Eur Cytokine Netw. 2002;13(1):55-65.

23. Sheng J G, Mrak R E, Griffin W S. Enlarged and phagocytic, but not primed, interleukin-1 alpha-immunoreactive microglia increase with age in normal human brain. Acta Neuropathol. 1998;95(3):229-34.

24. Itoh Y, Hayashi H, Miyazawa K, Kojima S, Akahoshi T, Onozaki K. 17 -Estradiol Induces IL-1 Gene Expression in Rheumatoid Fibroblast-Like Synovial Cells through Estrogen Receptor (ER) and Augmentation of Transcriptional Activity of Sp1 by Dissociating Histone Deacetylase 2 from ER The Journal of Immunology. 2007;178(5):3059-66. doi: 10.4049/jimmuno1.178.5.3059.

25. Boutin H, LeFeuvre R A, Horai R, Asano M, Iwakura Y, Rothwell N J. Role of IL-1α and IL-lb in Ischemic Brain Damage. J Neurosci. 2001;21(15):5528-34.

26. Lee B, Clarke D, Al Ahmad A, Kahle M, Parham C, Auckland L, Shaw C, Fidanboylu M, Orr A W, Ogunshola O, Fertala A, Thomas S A, Bix G J. Perlecan domain V is neuroprotective and proangiogenic following ischemic stroke in rodents. J Clin Invest. 2011;121(8):3005-23. doi: 10.1172/JCI46358. PubMed PMID: 21747167; PMCID: PMC3148740.

27. Bix G J, Gowing E K, Clarkson A N. Perlecan domain V is neuroprotective and affords functional improvement in a photothrombotic stroke model in young and aged mice. Transl Stroke Res. 2013;4(5):515-23. doi: 10.1007/s12975-013-0266-1. PubMed PMID: 24323378; PMCID: PMC3937769.

28. Banerjee M, Saxena M. Interleukin-1 (IL-1) family of cytokines: role in type 2 diabetes. Clin Chim Acta. 2012;413(15-16):1163-70. doi: 10.1016/j.cca.2012.03.021. PubMed PMID: 22521751.

29. Krishnan S M, Sobey C G, Latz E, Mansell A, Drummond G R. IL-lbeta and IL-18: inflammatory markers or mediators of hypertension? Br J Pharmacol. 2014;171(24):5589-602. doi: 10.1111/bph.12876. PubMed PMID: 25117218; PMCID: PMC4290704.

30. Saini M G, Bix G J. Oxygen-glucose deprivation (OGD) and interleukin-1 (IL-1) differentially modulate cathepsin B/L mediated generation of neuroprotective perlecan LG3 by neurons. Brain Res. 2012;1438:65-74. doi: 10.1016/j.brainres.2011.12.027. PubMed PMID: 22244880; PMCID: PMC3273646.

31. Pinteaux E, Trotter P, Simi A. Cell-specific and concentration-dependent actions of interleukin-1 in acute brain inflammation. Cytokine. 2009;45(1):1-7. doi: 10.1016/j.cyto.2008.10.008. PubMed PMID: 19026559.

32. Pringle A K, Niyadurupola N, Johns P, Anthony D C, Iannotti F. Interleukin-1^{˜} exacerbates hypoxia-induced neuronal damage, but attenuates toxicity produced by simulated ischaemia and excitotoxicity in rat organotypic hippocampal slice cultures. Neurosci Lett. 2001;305:29-32.

33. Bernardino L, Xapelli S, Silva A P, Jakobsen B, Poulsen F R, Oliveira C R, Vezzani A, Malva J O, Zimmer J. Modulator effects of interleukin-1beta and tumor necrosis factor-alpha on AMPA-induced excitotoxicity in mouse organotypic hippocampal slice cultures. J Neurosci. 2005;25(29):6734-44. doi: 10.1523/JNEUROSCI.1510-05.2005. PubMed PMID: 16033883.

34. Mattle H P, Kappeler L, Arnold M, Fischer U, Nedeltchev K, Remonda L, Jakob S M, Schroth G. Blood pressure and vessel recanalization in the first hours after ischemic stroke. Stroke. 2005;36(2):264-8. doi: 10.1161/01.STR.0000153052.59113.89. PubMed PMID: 15637309.

35. Fischer E, Marano M A, Barber A E, Hudson A, Lee K, Rock C S, Hawes A S, Thompson R C, Hayes T J, Anderson T D. Comparison between effects of interleukin-1 alpha administration and sublethal endotoxemia in primates. Am J Physiol. 1991;261(2.2):R442-52.

36. Maniskas M E, Bix G J, Fraser J F. Selective intra-arterial drug administration in a model of large vessel ischemia. J Neurosci Met. 2015;240:22-7.

37. Hassan A E, Chaudhry S A, Grigoryan M, Tekle W G, Qureshi A I. National trends in utilization and outcomes of endovascular treatment of acute ischemic stroke patients in the mechanical thrombectomy era. Stroke. 2012;43(11):3012-7. doi: 10.1161/STROKEAHA.112.658781. PubMed PMID: 22968467; PMCID: PMC3523170.

38. Chen J, Venkat P, Zacharek A, Chopp M. Neurorestorative therapy for stroke. Front Hum Neurosci. 2014;8:382. doi: 10.3389/fnhum.2014.00382. PubMed PMID: 25018718; PMCID: PMC4072966.

39. Banks W A, Ortiz L, Plotkin S R, Kastin A J. Human interleukin (IL) 1 alpha, murine IL-1 alpha and murine IL-1 beta are transported from blood to brain in the mouse by a shared saturable mechanism. J Pharmacol Exp Ther. 1991;259(3):988-96.

40. Diem R, Hobom M, Grotsch P, Kramer B, Bahr M. Interleukin-1 beta protects neurons via the interleukin-1 (IL-1) receptor-mediated Akt pathway and by IL-1 receptor-independent decrease of transmembrane currents in vivo. Mol Cell Neurosci. 2003;22(4):487-500; PMCID: 12727447.

41. Andre R, Wheeler R D, Collins P D, Luheshi G N, Pickering-Brown S, Kimber I, Rothwell N J, Pinteaux E. Identification of a truncated IL-18R13 mRNA: a putative regulator of IL-18 expressed in rat brain. J Neuroimmunol. 2003;145(1-2):40-5.

42. Roberts J, Kahle M P, Bix G J. Perlecan and the blood-brain barrier: beneficial proteolysis? Front Pharmacol. 2012;3:155. doi: 10.3389/fphar.2012.00155. PubMed PMID: 22936915; PMCID: PMC3425914.

43. Jin K L, Mao X O, Greenberg D A. Vascular endothelial growth factor: Direct neuroprotective effect in in vitro ischemia. PNAS. 2000;97(18):10242-7.

44. Rodgers K D, Sasaki T, Aszodi A, Jacenko O. Reduced perlecan in mice results in chondrodysplasia resembling Schwartz-Jampel syndrome. Hum Mol Genet. 2007;16(5):515-28. doi: 10.1093/hmg/dd1484. PubMed PMID: 17213231.

45. Abdulaal W H, Walker C R, Costello R, Redondo-Castro E, Mufazalov I A, Papaemmanouil A, Rothwell N J, Allan S M, Waisman A, Pinteaux E, Muller W. Characterization of a conditional interleukin-1 receptor 1 mouse mutant using the Cre/LoxP system. Eur J Immunol. 2016;46(4):912-8. doi: 10.1002/eji.201546075. PubMed PMID: 26692072.

46. Clarke D N, Al Ahmad A, Lee B, Parham C, Auckland L, Fertala A, Kahle M, Shaw C S, Roberts J, Bix G J. Perlecan Domain V induces VEGf secretion in brain endothelial cells through integrin alpha5betal and ERK-dependent signaling pathways. PLoS One. 2012;7(9):e45257. doi: 10.1371/journal.pone.0045257. PubMed PMID: 23028886; PMCID: PMC3444475.

47. Shindo A, Maki T, Mandeville E T, Liang A C, Egawa N, Itoh K, Itoh N, Borlongan M, Holder J C, Chuang T T, McNeish J D, Tomimoto H, Lok J, Lo E H, Arai K. Astrocyte-Derived Pentraxin 3 Supports Blood-Brain Barrier Integrity Under Acute Phase of Stroke. Stroke. 2016;47(4):1094-100. doi: 10.1161/STROKEAHA.115.012133. PubMed PMID: 26965847; PMCID: PMC4811738.

48. Roberts J, de Hoog L, Bix G J. Mice deficient in endothelial alpha5 integrin are profoundly resistant to experimental ischemic stroke. J Cereb Blood Flow Metab. 2015. doi: 10.1177/0271678X15616979. PubMed PMID: 26661237.

49. Maniskas M E, Roberts J M, Aron I, Fraser J F, Bix G J. Stroke neuroprotection revisited: Intra-arterial verapamil is profoundly neuroprotective in experimental acute ischemic stroke. J Cereb Blood Flow Metab. 2016;36(4):721-30. doi: 10.1177/0271678X15608395. PubMed PMID: 26661189; PMCID: PMC4821022.

50. Welsh C J R, Sapatino B V, Rosenbaum B A, Smith R. Characteristics of cloned cerebrovascular endothelial cells following infection with Theiler's virus I. Acute infection. J Neuroimmunol. 1995; 62(2): 119-25.

51. Welser-Alves J V, Boroujerdi A, Milner R. Isolation and culture of primary mouse brain endothelial cells. Methods Mol Biol. 2014;1135:345-56. doi: 10.1007/978-1-4939-0320-7_28. PubMed PMID: 24510877.

1. A method for treating ischemia in a subject, comprising:
administering to a subject in need thereof an effective amount of IL-1α thereby treating the ischemia, wherein the ischemia is caused by an ischemic event selected from cerebral ischemia and/or stroke.

2. The method of claim 1, wherein the IL-1α is administered during or after the onset of the ischemia.

3. The method of claim 1, wherein the IL-1α is administered about 0.5 to about 4 hours after the onset of the ischemia.

4. The method of claim 1, wherein the IL-1α is administered prior to the onset of ischemia.

5. The method of claim 1, wherein the treatment prevents the occurrence of an infarction.

6. The method of claim 1, wherein the treatment restores perfusion to organs and tissues.

7. The method of claim 1, wherein the administering step includes administering about 0.005 μg/kg to about 5 mg/kg of the IL-1α.

8. The method of claim 1, wherein the number of apoptopic cells, inflammation and/or newly divided cells is reduced.

9. The method of claim 1, wherein the administering is performed intravenously or intraarterially.

10. The method of claim 1, wherein the administering reduces infarct volume and/or peri-infarct expansion.

11. A method of reducing infarcts in a subject after ischemic stroke comprising: administering IL-1α to the subject.

12. The method of claim 10, further comprising administration of IL-1RA.

13. The method of claim 1 wherein the administration is acute.

14. The method of claim 10, wherein the administering increases perlecan, Cathepsin B mRNA, PTX3, or combinations thereof.

15. A method of treating ischemia comprising: administering intravenous IL-1α during the acute phase of injury.

16. The method of claim 15 wherein the administering sustains a low grade chronic inflammation.

17. The method of claim 15, wherein the dose of IL-1α is subpathological.

18. The method of claim 15, wherein the IL-1α is administered at about 0.05 μg/kg to about 5 mg/kg.

19. The method of claim 1, further comprising administration of IL-1RA.

20. The method of claim 1, wherein the administering increases perlecan, Cathepsin B mRNA, PTX3, or combinations thereof.
